SEQUENCE LISTING (2 sequences)

SEQ ID NO: 1 - DNA, 20 bp, Artificial Sequence
  Synthetic Construct
  misc_feature (1)..(20): Phosphorothioate backbone
tccatgacgttcctgatgct

SEQ ID NO: 2 - DNA, 20 bp, Artificial Sequence
  Synthetic Construct
  misc_feature (1)..(20): Phosphorothioate backbone
tccatgagcttcctgatgct

(19) United States
(12) Patent Application Publication
(10) Pub. No.: US 2022/0041682 A1
(43) Pub. Date: Feb. 10, 2022

(54) TLR9-TARGETED THERAPEUTICS

(71) Applicants: H. Lee Moffitt Cancer Center and Research Institute, Inc., Tampa, FL (US); ANP Technologies, Inc., Newark, DE (US)

(72) Inventors: Alan List, Tampa, FL (US); Sheng Wei, Tampa, FL (US); Ray Yin, Wilmington, DE (US); Jing Pan, Newark, DE (US)

(21) Appl. No.: 17/281,411

(22) PCT Filed: Nov. 13, 2019
(86) PCT No.: PCT/US19/61220

§ 371 Date: Mar. 30, 2021

Related U.S. Application Data
(60) Provisional application No. 62/760,362, filed on Nov. 13, 2018.

(51) Int. Cl. C07K 14/705; A61K 47/64; A61K 47/66; C12N 15/117

(52) U.S. Cl. CPC C07K 14/705; C12N 15/117; A61K 47/66; A61K 47/64

(57) ABSTRACT

Disclosed are compositions and methods for targeted treatment of TLR9-expressing cancers. In particular, disclosed herein are molecules or conjugates containing a TLR9 targeting ligand, such as a CpG oligodeoxynucleotide, and a cytotoxic nanoparticle that targets TLR9-expressing malignant cells. Also disclosed is a pharmaceutical composition comprising a molecule disclosed herein in a pharmaceutically acceptable carrier. Also disclosed is a method for treating a TLR9-positive cancer in a subject that involves administering to the subject a therapeutically effective amount of a disclosed pharmaceutical composition.

### CROSS-REFERENCE TO RELATED APPLICATIONS

This application claims benefit of U.S. Provisional Application No. 62/760,362, filed Nov. 13, 2018, which is hereby incorporated herein by reference in its entirety.

### SEQUENCE LISTING

This application contains a sequence listing filed in electronic form as an ASCII.txt file entitled “320301_2040_Sequence_Listing_ST25” created on Nov. 13, 2019. The content of the sequence listing is incorporated herein in its entirety.

### TECHNICAL FIELD

This application relates generally to compositions and methods for treating cancers, such as myelodysplastic syndromes (MDS).

### BACKGROUND

Myelodysplastic syndromes (MDS) are hematopoietic stem cell malignancies with a rising prevalence owing to the aging of the American population. MDS comprise a group of malignant hematologic disorders associated with impaired erythropoiesis, dysregulated myeloid differentiation and increased risk for acute myeloid leukemia (AML) transformation. The incidence of MDS is increasing with 15,000 to 20,000 new cases each year in the United States and large numbers of patients requiring chronic blood transfusions. Ineffective erythropoiesis remains the principal therapeutic challenge for patients with more indolent subtypes, driven by a complex interplay between genetic abnormalities intrinsic to the MDS clone and senescence dependent inflammatory signals within the bone marrow (BM) microenvironment. Although three agents are approved for the treatment of MDS in the United States (US), lenalidomide (LEN) represents the only targeted therapeutic. Treatment with LEN yields sustained red blood cell transfusion independence accompanied by partial or complete resolution of cytogenetic abnormalities in the majority of patients with a chromosome 5q deletion (del5q), whereas only a minority of patients with non-del5q MDS achieve a meaningful response, infrequently accompanied by cytogenetic improvement. Although responses in patients with del5q MDS are relatively durable, lasting a median of 2.5 years, resistance emerges over time with resumption of transfusion dependence.

The available effective treatment options for patients with non-del (5q) is limited. Notably, MDS cases grow year over year due the increase in the American aging population and its combination. Frequently they are misdiagnosed leading to failure to treat serious infections or the wasting of expensive treatment and precious resources. Once a proper diagnosis is made patients have to rely on frequent blood transfusion and non-specific chemotherapy which have severe side effects and have limited benefit for patients with non-del (5q). The lack of effective treatment on MDS patients without del (5q) contributes to the enormous burden of this disease on both patient and caregivers and increases the risk of AML transformation. Therefore, there is definitely a need to develop a specific targeted therapeutic in this patient population

### SUMMARY

Compositions and methods are disclosed for targeted treatment of cancer or cancer-stem cells with extracellular TLR9 expression, such as primary human MDS progenitors, hematopoietic stem cell (HSC), and liver cancers. In particular, molecules containing TLR9 targeting ligands that target nanoparticles to TLR9-expressing malignant cells are disclosed.

In some embodiments, the molecule comprises a TLR9 targeting ligand conjugated to a cytotoxic nanoparticle, e.g. via a bivalent linker. In particular embodiments, the nanoparticle/polymer comprises a mean diameter small enough that it is not engulfed by macrophages. For example, the nanoparticle/polymer can have a mean diameter of about 1 to about 50 nm, including about 1.5 to about 10 nm. In some cases, the cytotoxic nanoparticle/polymer is loaded with a cytotoxic agent, such as those disclosed above. In some cases, the cytotoxic nanoparticle/polymer comprises cationic polymer including branched polymer/dendrimer (both symmetrical and asymmetrical), a homopolymer comprising both linear and branch architectures, or a copolymer such as block copolymer, graft copolymer, random copolymer, and/or a combination thereof. This cationic structure of the nanoparticle/polymer can be cytotoxic once internalized by a cell, but non-toxic outside the cell.

In a variety of aspects, the TLR9 targeting ligand is an unmethylated CpG oligodeoxynucleotide, or an analogue or derivative thereof that binds TLR9. For example, in some cases, the TLR9 ligand comprises the CpG sequence T*C*C*A*T*G*A*C*G*T*T*C*C*T*G*A*T*G*C*T* (SEQ ID NO:1) that is modified with thioate linkages (*=phosphorothioate linkage).

In some embodiments, the bivalent linker is a C6 amino-SMCC-Cys linker. In some embodiments, the linker is produced using click chemistry reactions. The click chemistry approach was originally conceived as a method to rapidly generate complex substances by joining small subunits together in a modular fashion. Various forms of click chemistry reaction are known in the art, such as the Huisgen 1,3-dipolar cycloaddition copper catalyzed reaction, which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

Also disclosed is a pharmaceutical composition comprising a molecule disclosed herein in a pharmaceutically acceptable carrier. Also disclosed is a method for treating a TLR9-positive cancer in a subject that involves administering to the subject a therapeutically effective amount of a disclosed pharmaceutical composition.

The cancer of the disclosed methods can be any TLR9-positive cell in a subject undergoing unregulated growth, invasion, or metastasis. Thus, the cancer can be a sarcoma, lymphoma, leukemia, carcinoma, blastoma, or germ cell tumor. A representative but non-limiting list of cancers include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some cases, the cancer comprises a myelodysplastic syndrome (MDS). For example, the cancer can be non-del (5q) MDS. FIG. 4 identifies other cancers, such as lung, liver, and breast cancers, that have increased TLR9 expression. FIG. 5 shows TLR9 protein expression in a variety of tumor tissues. For example, cancers of the skin, esophagus, colon, rectum, liver, lung, and uterus have been shown to have increased TLR9 protein expression. In some cases, the method further involves assaying a biopsy sample from the subject for TLR9 expression prior to treatment.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows synthesis path of linkage using Click-IT chemistry.

FIG. 2 shows sequences of a phosphorothioated CpG linked and peptide sequences and C6 amino-SMCC-Cys linker strategy.

FIG. 3 shows example lytic peptide 3D structure and structure of scrambled control used.

FIG. 4 is a box plot showing TLR9 overexpression in a variety of tumors. The box plot represents the 25th to 75th percentile (the box) with the median represented by the black line in the box. The outliers are in circles represent the median absolute deviation (2 SD is about the same).

FIG. 5 shows results of tissue microarray (TMA) slides being stained with anti-TLR9 antibody. The top graph shows the tabulated data for the cores in the control TMA and a representative picture from one of the cores demonstrating the lack of brown coloration. The only positive core in that slide was inflamed tonsils which serve as a positive control. The second graph shows the tabulated results from the multi-tumor TMA (48 cases of 15 cancers) showing varied levels of TLR9 positive staining. The picture represents the core for a melanoma case that had heavy brown staining as a representative figure.

FIG. 6 shows an example CpG-linked nanoparticle. FIG. 6A shows CpG-linked dendrimer nanoparticles. FIG. 6B shows CpG-linked biodegradable polymer nanoparticles. Each circle represents one or more biodegradable bonds. Not all polymer components, combinations, variations or linked CpG molecules are shown.

FIG. 7 shows increased expression in the MDS cell line SKM1 by flow cytometric analysis.

FIG. 8 shows gating strategy to check for apoptosis of SKM1 cells after treatment with CpG-dendrimers. Cells were gated for singlets from where the populations demonstrating early apoptosis (Annexin V+7AAD−, left top gate), late apoptosis (Annexin V+7AAD+, right top gate) or necrosis (Annexin V−7AAD+, right bottom gate).

FIG. 9 shows testing of SKM1 cells after treatment with different CpG-linked payloads with nanoparticles. Three distinct conjugates were tested (labeled 1, 2 or 3) at three separate doses 0.05, 0.1 or 0.5 μM before assessing by flow cytometry for cell death by Annexin V/7AAD.

FIG. 10 shows gating strategy for the images shown in FIGS. 11 and 12

FIG. 11 shows testing of SKM1 cells after treatment with different CpG-linked payloads with nanoparticles. Three distinct conjugates were tested (labeled 1, 2 or 3) at three separate doses 0.05, 0.1 or 0.5 uM before assessing by flow cytometry for cell death by 7AAD.

FIG. 12 shows testing of SKM1 cells after treatment with different CpG-linked payloads with nanoparticles. Three distinct conjugates were tested (labeled 1, 2 or 3) at three separate doses 0.05, 0.1 or 0.5 uM before assessing by flow cytometry for cell death by Annexin V.

FIG. 13 shows TLR9 expression in HepG2, Huh7, and Huh7.5 cells.

FIG. 14 shows quantification of SKM1 cells imaged continuously while in culture in an Incucyte microscope at brightfield after treatment with either control (unconjugated) nanoparticle versus CpG-linked nanoparticles (Conjugates 1, 2 and 3) at the doses 0.05, 0.1 and 0.5 uM for 72 hours.

FIG. 15 shows gating strategy for the images shown in FIGS. 16, 17 and 18

FIG. 16 shows testing of primary MDS BM cells after treatment with different CpG-linked payloads with nanoparticles. Three distinct conjugates were tested (labeled 1, 2 or 3) at three separate doses 0.05, 0.01 or 0.005 uM before assessing by flow cytometry for cell death by Annexin V/7AAD in gated CD34 cells.

FIG. 17 shows decrease of TLR9+CD34+ cells in a primary MDS BM treated with various CpG-dendrimer (full generation 5 polyamidoamine (PAMAM) dendrimer, E5) conjugates (Conjugates #1, #2, and #3).

FIG. 18 shows testing of primary MDS BM cells after treatment with different CpG-linked cationic PAMAM dendrimer payloads. Three distinct conjugates were tested (labeled 1, 2 or 3) at three separate doses 0.05, 0.01 or 0.005 uM before assessing by flow cytometry for cell death by Annexin V/7AAD in gated CD71 cells.

FIG. 19 shows two separate MDS BM primary specimens were treated with CpG conjugated with siRNA targeting BmaI1 sequences 1 and 2 (two different sequences) or their combination, cultured for 48 hours before reculturing in methylcellulose media supplemented with growth factors for 14 days. At that point hematopoietic colonies were counted for BFU-E and CFU-GM.

FIG. 20 shows an MDS BM primary specimen was treated with CpG conjugated with siRNA targeting BmaI1 sequences 1 and 2 (two different sequences) or their combination, cultured for 48 hours before cytospinning into a slide. Cells were then stained for BmaI1 expression and DAPI for nuclear staining. Slides were pictured in a confocal microscope.

FIG. 21 shows three separate MDS BM primary specimens were treated with CpG conjugated with lytic peptide, cultured for 48 hours before reculturing in methylcellulose media supplemented with growth factors for 14 days. At that point hematopoietic colonies were counted for BFU-E and GFU-GM.

FIG. 22 shows gating strategy to check for apoptosis of SKM1 cells after treatment with CpG-dendrimers. Cells were gated for singlets from where the populations demonstrating early apoptosis (Annexin V+7AAD−, left top gate), late apoptosis (Annexin V+7AAD+, right top gate) or necrosis (Annexin V−7AAD+, right bottom gate). SKM-1 cells treated with CpG-Dendrimer for 48 h. Cells stained with L/D dye near infrared, TLR9-APC Anenixin V-FITC and 7AAD. Flow cytometry was then performed.

FIG. 23 shows testing of SKM1 cells after treatment with different CpG-linked cationic PAMAM dendrimer payloads. Three distinct conjugates were tested (labeled 1, 2 or 3) at three separate doses 0.05, 0.1 or 0.5 μM before assessing by flow cytometry for side-scatter and forward scatter.

FIG. 24 shows testing of SKM1 cells after treatment with different CpG-linked cationic PAMAM dendrimer payloads. Three distinct conjugates were tested (labeled 1, 2 or 3) at three separate doses 0.05, 0.1 or 0.5 uM before assessing by flow cytometry for cell death by Annexin V/7AAD.

FIG. 25 is a schematic representation of xenograft model testing CpG-dendrimer against TLR9+ solid tumor. Briefly, NSG mice were injected with 10×10\hat{ }6 HepG2 cells and allowed to grow for 4 weeks until they reached 40 mm3 in size for MRI detection. At that point animals were separated into two groups: 1) receiving unconjugated-Dendrimer 2) receiving CpG-linked dendrimer. Injections were performed by giving 5 ug/mouse intra peritoneally and 5 ug/mouse into three separate peritumoral injections every other day for 4 weeks with weekly MRI measurements.

FIG. 26A shows average tumor volume from the pilot xenograft study using TLR9+ HepG2 cells in NSG mice from weekly measurements. FIG. 26B shows same data as in FIG. 26A but presented as a ratio of the tumor volume of that day against the initial tumor volume. Error bars represent the SEM of n=5 and the * indicate p<0.005 measured by paired student t-test.

FIG. 27 shows representative MRI scanning of mice from CpG-dendrimer versus Dendrimer-only groups.

FIG. 28 are representative excised tumors from mice treated with CpG-dendrimer, CpG only and Dendrimer only groups. The CpG only group only had one mouse and therefore not added to the graph tabulations of tumor growth shown in the previous pictures.

FIG. 29 shows excised tumor weight and mouse weight without tumor. NO changes in the mice size were observed although there were tumor variations.

FIG. 30 shows TLR9 expression on luciferin-inducible HepG2 cells after in vivo treatment.

FIG. 31 shows CpG-dendrimer potential effects on MDSC.

FIG. 32 shows CpG-dendrimer potential effects on B cells.

FIG. 33 shows CpG-dendrimer potential effects on DCs.

FIG. 34 CpG-dendrimer potential effects on T cells.

FIG. 35 shows CpG, dendrimer or CpG-dendrimer treatment in vivo does not have specific associated pathologies.

FIG. 36 shows HepG2 xenograft model: Tumor pathology descriptions (representative H&E from each group).

FIGS. 37A and 37B shows effects of CpG-dendrimer in vivo in spleen and bone marrow. Intravenous injection (2nd time) total of three weeks

FIGS. 38A and 38B show results of a SKM-1 cell viability (MTS) assay after TLR9 targeted therapy using CpG-E3 conjugates (1:1) (FIG. 38A) and CpG-E5 conjugates (1:2) (FIG. 38B) in vitro.

FIGS. 39A to 39G show results of targeting TLR9 HSPC and CMP in mice after treatment with CpG-dendrimer. FIG. 39A shows the timeline for wildtype (WT) and S100A9 transgenic (Tg) mice injection with 10 μg CpG or CpG-Den, twice per week. FIGS. 39B and 39C show RBC (FIG. 39B) and HBG (FIG. 39C) in treated mice at each time point.

FIGS. 39D to 39G show percentage of bone marrow (FIGS. 39D and 39E) or spleen cells (FIGS. 39F and 39G) that are Lin⁻CD16/32^{lo}c-Kit⁺Sca-1⁻TLR9⁺ (FIG. 39D or 39F) or Lin⁻ CD16/32⁻c-Kit⁺ TLR9⁺ (FIG. 39E or 39G).

FIGS. 40A to 40C show Cy3 fluorescence after intravenous injection of CpG-dendrimer. FIG. 40A shows the injection scheme. FIG. 40B shows in vivo fluorescence using an Bruker intra vital imager. FIG. 40C shows fluorescence at 0, 2, and 24 hours in spleen, liver, and bone.

FIG. 41 shows fluorescence in an IP cell SKM1 model 1 week after treatment with Biosynthesis and IDT CpG-dendrimers.

FIG. 42 shows CpG-dendrimer pathology in kidney, liver, lung, and spleen.

FIG. 43 shows SKM1 cells treated with CpG, dendrimer, or CpG-dendrimer for 24 hours before monitoring cell death. The competition experiment were performed same way but pretreated with unlabeled CpG for 4 hours before adding CpG-dendrimer.

FIG. 44 shows an example Poly(amidoamine) (PAMAM) dendrimer, which can be adjusted to have different sizes, such as two or more concentric dendritic layers, such as 3 layers, 4 layers or 5 layers, also referred to as E3, E4 or E5 dendrimer, respectively.

FIG. 45 shows an example poly-L-lysine (PLL) dendrimer.

FIG. 46 shows survival over time of HepG2 xenograft mice after treatment with control, CpG-Den, CpG, or Den.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “CpG motif” refers to a nucleotide sequence, which contains unmethylated cytosine-guanine dinucleotide linked by a phosphate bond.

The term “CpG oligodeoxynucleotide” or “CpG ODN” refers to an oligodeoxynucleotide comprising at least one CpG motif and that binds TLR9.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

The term “isolated nucleic acid” refers to a polynucleotide of genomic, cDNA, synthetic, or natural origin or some combination thereof, which (1) is not associated with the cell in which the “isolated nucleic acid” is found in nature, or (2) is operably linked to a polynucleotide to which it is not linked in nature.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” is meant a molecule that is produced as a result of transcription of a gene. Gene products include RNA molecules transcribed from a gene, as well as proteins translated from such transcripts.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “polymer”, “polymers” as used herein refers to homopolymer polymerized from substantially the same monomer, copolymer polymerized from different monomers, or a combination thereof. The polymer can be linear polymer, branched polymer, dendrimer, symmetrically branched polymer, asymmetrically branched polymer, blocked copolymer, graft copolymer, random copolymer, or a combination thereof. The polymer can be polymerized from modified or unmodified monomers, macromonomers, another polymer or polymers, or a combination thereof. The polymer can be polymerized from condensation polymerization, free radical polymerization, living polymerization, chain addition polymerization, reversible addition-fragmentation chain transfer (RAFT) polymerization, atom transfer radical polymerization (ATRP), or any other polymerization processes.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

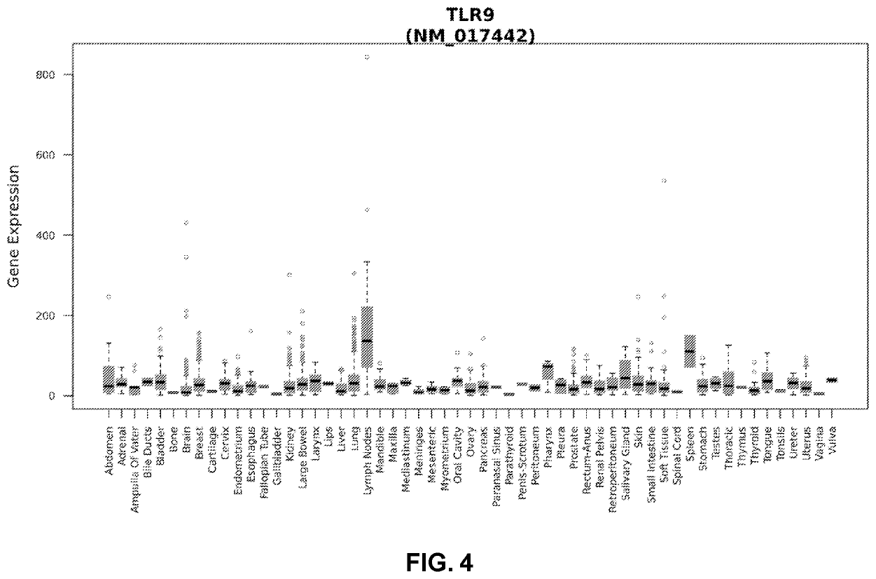

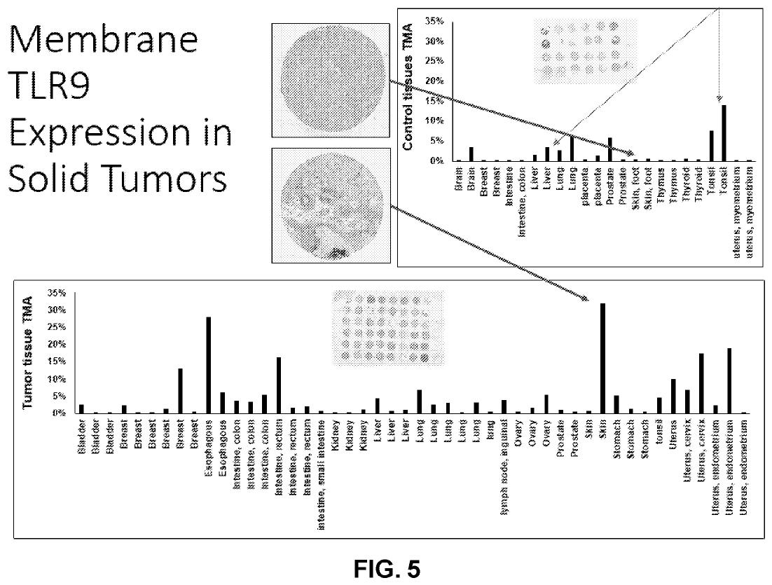

Disclosed are compositions and methods for targeting TLR9-expressing cancers, such as primary human MDS hematopoietic stem and progenitor cells (HSPC). FIG. 4 identifies other cancers, such as lung and breast cancers, that have increased TLR9 expression. FIG. 5 shows TLR9 protein expression in a variety of tumor tissues. For example, cancers of the skin, esophagus, colon, rectum, liver, lung, and uterus have been shown to have increased TLR9 protein expression.

In particular, molecules containing TLR9 targeting ligands that target cytotoxic agents to TLR9-expressing malignant cells are disclosed. Therefore, the molecule can comprise a TLR9 targeting ligand (“TTL”) and a cytotoxic nanoparticle disclosed herein. For example, the TTL and cytotoxic agent can be joined by a bivalent linker. In some embodiments, the molecule comprises a TTL conjugated to any payload, such as a diagnostic or therapeutic agent, via a bivalent linker disclosed herein. Non-limiting examples of payloads include therapeutic and diagnostic agents, as well as nanoparticles encapsulating a therapeutic or diagnostic agent. For example, therapeutic agents include cytotoxic compounds, lytic peptides, cytotoxic nanoparticles, cationic toxic lipid moieties, radioactive isotypes, or other chemical compounds.

### TLR9 Targeting Ligand

The TTL can in some embodiments be a CpG oligodeoxynucleotide, such as an unmethylated CpG oligodeoxynucleotide, or an analogue or derivative thereof that binds TLR9. CpG oligodeoxynucleotides (or CpG ODN) are short single-stranded synthetic DNA molecules that contain a cytosine triphosphate deoxynucleotide followed by a guanine triphosphate deoxynucleotide. The “p” refers to the phosphodiester link between consecutive nucleotides, although some ODN have a modified phosphorothioate (PS) backbone instead. When these CpG motifs are unmethlyated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed internally only in B cells and plasmacytoid dendritic cells (pDCs) in humans and other higher primates. However, extracellular expression of this receptor only happens in certain pathologies. Moreover, MDS progenitors, and in particular MDS stem cells (HSC), overexpress Toll-like receptor (TLR)-9 extracellularly, permitting development of a targeting approach using unmethylated CpG oligonucleotides linked to bioactive payloads for cellular delivery.

Synthetic CpG ODN differ from microbial DNA in that they have a partially or completely phosphorothioated (PS) backbone instead of the typical phosphodiester backbone and a poly G tail at the 3′ end, 5′ end, or both. PS modification protects the ODN from being degraded by nucleases such as DNase in the body and poly G tail enhances cellular uptake. The poly G tails form intermolecular tetrads that result in high molecular weight aggregates. Numerous sequences have been shown to stimulate TLR9 with variations in the number and location of CpG dimers, as well as the precise base sequences flanking the CpG dimers. This led to the creation of five unofficial classes or categories of CpG ODN based on their sequence, secondary structures, and effect on human peripheral blood mononuclear cells (PBMCs). The five classes are Class A (Type D), Class B (Type K), Class C, Class P, and Class S.

In some embodiments, the TTL comprises an antibody. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

### Bivalent Linker

The bivalent linker can be any molecule suitable to link a compound, polypeptide, or nucleic acid to a TTL (e.g., CpG ODN). Methods and compositions for conjugating biomolecules, such as polynucleotides, are disclosed in G. T. Hermanon, Bioconjugate Techniques (2^{nd} ed.), Academic Press (2008), which is incorporated by reference in its entirety for the teaching of these techniques.

In some embodiments, the bivalent linker is a non-nucleotidic linker. As used herein, the term “non-nucleotidic” refers to a linker that does not include nucleotides or nucleotide analogs. Typically, non-nucleotidic linkers comprise an atom such as oxygen or sulfur, a unit such as C(O), C(O)NH, SO, SO₂, SO₂NH, or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, SS, S(O), SO₂, N(R¹)₂, NR¹, C(O), C(O)O, C(O)NH, —OPO₂O—, cleavable linking group, substituted or unsubstituted aryl, substituted or unsubstituted heteroaryl, substituted or unsubstituted heterocyclic; where R′ is hydrogen, acyl, aliphatic or substituted aliphatic. The polymeric linkers, including, but not limited to, polyethyleneoxide (PEO), polyethylene glycol (PEG), poly (2-methyloxazoline), poly (2-ethyloxazoline), etc., such as those described in US Patent Application 20160024252, can also be used.

In some embodiments, the bivalent linker comprises at least one cleavable linking group, i.e. the linker is a cleavable linker. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the linked components after transport to the desired target. The intended nature of the conjugation or coupling interaction, or the desired biological effect, will determine the choice of linker group. For example, the bivalent linker can comprise a photocleavable PC linker. In some embodiments, the oligonucleotide is cleavable by dicer to produce isolate individual siRNA from the oligonucleotide.

Additional examples of linkers include Hexanediol, Spacer 9, Spacer 18, 1′,2′-Dideoxyribose (dSc), and I-Linker.

In some embodiments, the linker comprises m-Maleimidobenzyol-N-hydroxysuccinimide ester (MBS), Sulfo-m-maleimidobenzyol-N-hydroxysuccinimide ester (sulfo-MBS), or Sulfosuccinimidyl-4-(N-maleimidomethyl)cyclohexane-1-carboxylate (SMCC).

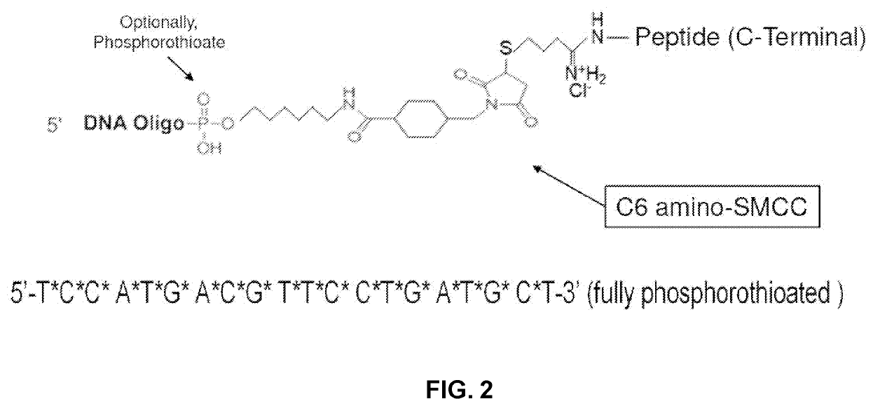

In some embodiments, the linker is a C6 amino-SMCC-Cys linker as shown in FIG. 2.

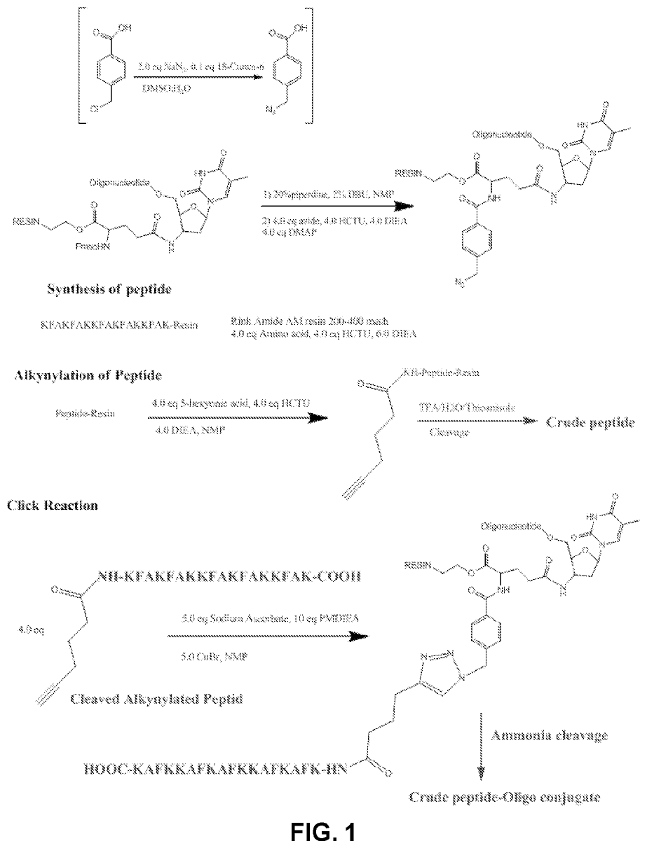

In some embodiments, the linker is produced using click chemistry reactions, as shown in FIG. 1. The click chemistry approach was originally conceived as a method to rapidly generate complex substances by joining small subunits together in a modular fashion. Various forms of click chemistry reaction are known in the art, such as the Huisgen 1,3-dipolar cycloaddition copper catalyzed reaction, which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

### Polymer and Nanoparticle

The cytotoxic nanoparticle can comprise a cationic polymer and a TLR ligand (e.g. CpG) disclosed herein linked to the cationic polymer via the bivalent linker disclosed above and herein. The cationic polymer can comprise a linear polymer, a symmetrically branched polymer/dendrimer, an asymmetrically branched polymer/dendrimer, a linear and branched hybrid homopolymer, a copolymer such as a block copolymer, a graft copolymer, a random copolymer, or a combination thereof, wherein the CpG is covalently conjugated to said cationic polymer via the bivalent linker.

Cytotoxicity can generally be modulated by adjusting the sizes (molecular weight), branching, functional groups, cationic components, or a combination thereof. For example, a dendritic polymer can be adjusted to have different sizes, such as two or more concentric dendritic layers (generations), such as 3 layers, 4 layers or 5 layers (generations), also referred to as E3, E4 or E5 dendrimer, respectively. In further examples, a 3 or 5-layer PAMAM or PEI dendrimer, also referred to as E3 or E5 PAMAM or G3 or G5 PEI dendrimer, can be suitable.

In another example, a dendritic polymer can be adjusted to have symmetrical (equal length) branches and branch junctures or asymmetric branches (non-equal branch lengths).

In yet another example, functional groups, such as those including, but not limited to, amino (e.g., primary, secondary, tertiary and quaternary amino groups), carboxyl, ester, aliphatic, aromatic, silicon containing, fluorine containing, sulfur containing groups, etc., can be adjusted or derived from polyamidoamine (PAMAM)-based branched polymers and dendrimers described in U.S. Pat. Nos. 4,435,548; 4,507,466; 4,568,737; 4,587,329; 5,338,532; 5,527,524; and 5,714,166; from polyethyleneimine (PEI) dendrimers, such as those disclosed in U.S. Pat. No. 4,631,337; polypropyleneimine (PPI) dendrimers, such as those disclosed in U.S. Pat. Nos. 5,530,092; 5,610,268; and 5,698,662; and others, as described, for example, in, “Dendritic Molecules,” edited by Newkome et al., VCH Weinheim, 1996, “Dendrimers and Other Dendritic Polymers,” edited by Frechet & Toroalia, John Wiley & Sons, Ltd., 2001; and U.S. Pat. No. 7,754,500. Other functional groups, such as bioactive payloads or CpG can also be used for targeted delivery to enhance target-specific cytotoxicity.

In other examples, a polymer can have flexible and diversifying cationic components. In general, larger molecular weight of a dendritic polymer can help to enhance efficiency of delivery of bioactive agent into biosystems, but generally can have more cytotoxicity. Smaller cationic component can help to reduce cytotoxicity. In one embodiment, a dendritic polymer can be adjusted to have different cationic components, such as a polymerized polyethyleneimine (PEI), a propylethyleneimine (PPI), or a combination thereof, to have different electric cationic charges or charge density to suit various uses.

In further embodiments, charges of a dendritic polymer can further be adjusted by using acid, base, buffer, or a combination thereof. In some examples, a polymer can have positive charge under acidic or neutral pH conditions to affiliate with a negatively charged bioactive agent, such as a nucleic acid, an RNA, a DNA, an oligodeoxynucleotide and the like to produce a bioactive composition. In another example, a dendritic polymer can have neutral or negative charge under basic pH conditions to affiliate with a positively charged bioactive agent to produce a bioactive composition.

Symmetrically branched polymers (SBPs) are a class of polymers such as dendritic polymers (also referred to as dendrimers herein), including Starburst dendrimers (or Dense Star polymers) and Combburst dendrigrafts (or hyper comb-branched polymers), that have: (a) a well-defined core molecule, (b) at least two concentric dendritic layers (generations) with symmetrical (equal length) branches and branch junctures and (c) optionally, exterior surface groups, such as those including, but not limited to, amino, carboxyl, ester, aliphatic, aromatic, silicon containing, fluorine containing, sulfur containing groups, etc., derived from polyamidoamine (PAMAM)-based branched polymers and dendrimers described in U.S. Pat. Nos. 4,435,548; 4,507,466; 4,568,737; 4,587,329; 5,338,532; 5,527,524; and 5,714,166. Other examples include polyethyleneimine (PEI) dendrimers, such as those disclosed in U.S. Pat. No. 4,631,337; polypropyleneimine (PPI) dendrimers, such as those disclosed in U.S. Pat. Nos. 5,530,092; 5,610,268; and 5,698,662; Frechet-type polyether and polyester dendrimers, core shell tectodendrimers and others, as described, for example, in, “Dendritic Molecules,” edited by Newkome et al., VCH Weinheim, 1996, “Dendrimers and Other Dendritic Polymers,” edited by Frechet & Toroalia, John Wiley & Sons, Ltd., 2001; and U.S. Pat. No. 7,754,500.

Combburst dendrigrafts are constructed with a core molecule and concentric layers with symmetrical branches through a stepwise synthetic method. In contrast to dendrimers, Combburst dendrigrafts or polymers are generated with monodisperse linear polymeric building blocks (U.S. Pat. Nos. 5,773,527; 5,631,329 and 5,919,442). Moreover, the branch pattern is different from that of dendrimers. For example, Combburst dendrigrafts form branch junctures along the polymeric backbones (chain branches), while Starburst dendrimers often branch at the termini (terminal branches). Due to the living polymerization techniques used, the molecular weight distributions (Mw/Mn) of those polymers (core and branches) often are narrow. Thus, Combburst dendrigrafts produced through a graft-on-graft process are well defined with Mw/Mn ratios often approaching 1.

SBPs, such as dendrimers, are produced predominantly by repetitive protecting and deprotecting procedures through either a divergent or a convergent synthetic approach. Since dendrimers utilize small molecules as building blocks for the cores and the branches, the molecular weight distribution of the dendrimers often is defined. In the ease of lower generations, a single molecular weight dendrimer often is obtained. While dendrimers often utilize small molecule monomers as building blocks, dendrigrafts use linear polymers as building blocks.

Asymmetrically branched polymers (ABPs) are, particularly asymmetrically branched dendrimers or regular ABP (reg-ABP), often possess a core, controlled and well-defined asymmetrical (unequal length) branches and asymmetrical branch junctures as described in U.S. Pat. Nos. 4,289,872; 4,360,646; and 4,410,688.

A random asymmetrically branched polymer (ran-ABP) possesses: a) no core, b) functional, groups both at the exterior and in the interior, c) random/variable branch lengths and patterns (i.e., termini and chain branches), and d) unevenly distributed interior void spaces.

The synthesis and mechanisms of ran-ABPs, such as those made from randomly branched PEI, were reported by Jones et al., J. Org. Chem. 9, 125 (1944), Jones et al., J. Org. Chem. 30, 1994 (1965) and Dick et al., J. Macromol. Sci. Chem., A4 (6), 1301-1314, (1970)), while the synthesis of linear PEI was reported by Tomalia, et. al. on Macromolecules. 24: 1435-1438. Ran-ABP, such as those made of POX, i.e., poly(2-methyloxazoline) and poly(2-ethyloxazoline), was reported by Litt (J. Macromol. Sci. Chem. A9(5), 703-727 (1975)) and Warakomski (J. Polym. Sci. Polym. Chem. 28, 3551 (1990)). The synthesis of ran-ABP's often can involve a one-pot divergent or a one-pot convergent method.

Homopolymer refers to a polymer or a polymer backbone composed of the same repeat unit, that is, the homopolymer is generated from the same monomer (e.g., PEI linear polymers, POX linear polymers, PEI dendrimers, polyamidoamine (PAA) dendrimers or POX dendrigrafts and randomly ranched polymers). The monomer can be a simple compound or a complex or an assemblage of compounds, such as macromonomers or oligomers, where the assemblage or complex can be the repeat unit in the homopolymer. One or more of the monomer or complex monomer components can be modified, substituted, derivatized and so on, for example, modified to carry a functional group. Such molecules are homopolymers for the purposes of the instant disclosure as the backbone is composed of a single simple or complex monomer.

Dendronized polymers are linear polymer backbones having repeat dendron units attached thereon. Linear polymer backbones comprising biodegradable bonds, such as polypeptides, such as poly-L-Lysine, poly-L-Glutamic acid, Polyaspartic acid (PASA), or a combination thereof, polysaccharides can be suitable. Each of the dendron units can be branched, tree-like fragments. For example, a branched polyethyleneimine (PEI) or polypropyleneimine (PPI) can be suitable as a dendron unit.

Polymers disclosed in U.S. Pat. Nos. 8,597,653, 8,591,904 and US Patent Applications US2017/0066879, US2017/0024252 and US2014/0314664 can be suitable.

The cationic polymer can also comprise biodegradable polymers. The cytotoxic nanoparticle can comprise a biodegradable polymer comprising a biodegradable polymeric backbone comprising at least a biodegradable bond and two or more cationic polymer components, wherein each of the cationic polymer components is attached to the biodegradable polymeric backbone covalently separated by at least one of the biodegradable bond, wherein the CpG is covalently conjugated to the biodegradable polymer via the bivalent linker.

In one example, the biodegradable polymeric backbone can comprise polymerized amino acids, such as polymerized lysine, polymerized glutamic acid, polymerized aspartic acid, or a combination thereof. The cationic polymer components can be attached to adjacent amino acids in the backbone separated by one of the biodegradable bond. The cationic polymer components can also be attached to amino acids in the backbone separated by two or more of the biodegradable bonds.

The biodegradable bond can be a peptide bond, a reducible disulfide bond, glycosidic bond, or a combination thereof. The glycosidic bonds can be an α-1,6 glycosidic bonds, α-1,3 bonds, α-1,4 or α-1,6 bonds, or a combination thereof. The peptide bond and the glycosidic bond, or a combination thereof, can be preferred. The biodegradable bond can be cleaved or degraded by enzymes, such as peptidase, glycoside hydrolases, such as cellulase, hemicellulase, amylase, viral neuraminidases, mannosidases, or a combination thereof. The biodegradable bond can also include the bonds that can be cleaved or degraded by non-enzymatic cleavage, such as, but not limited to, acid hydrolysis such as under low pH conditions.

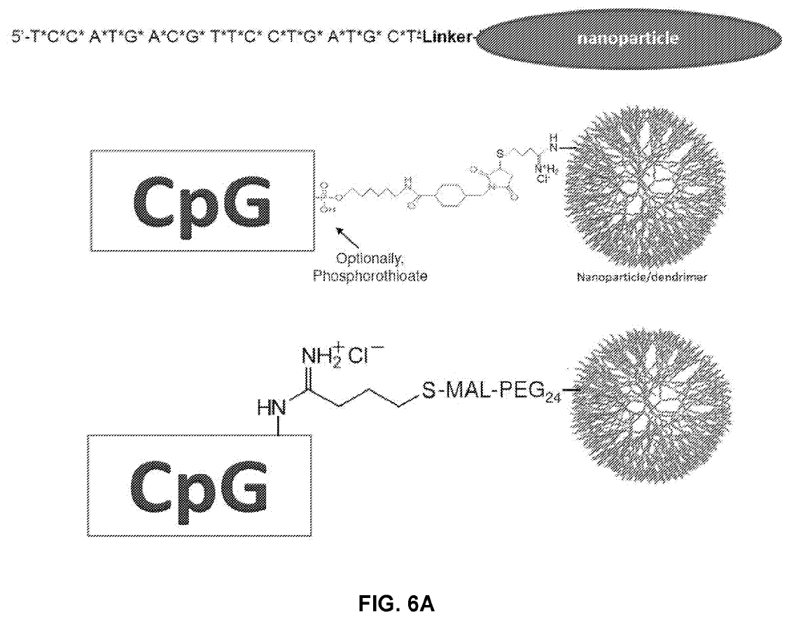

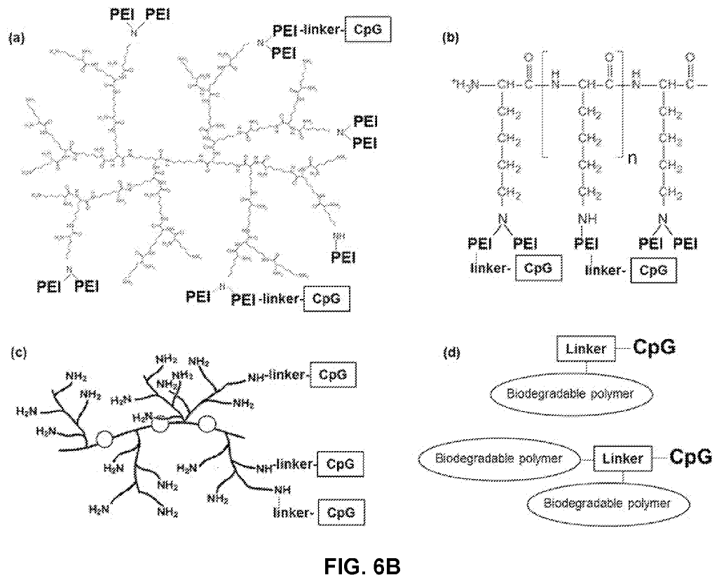

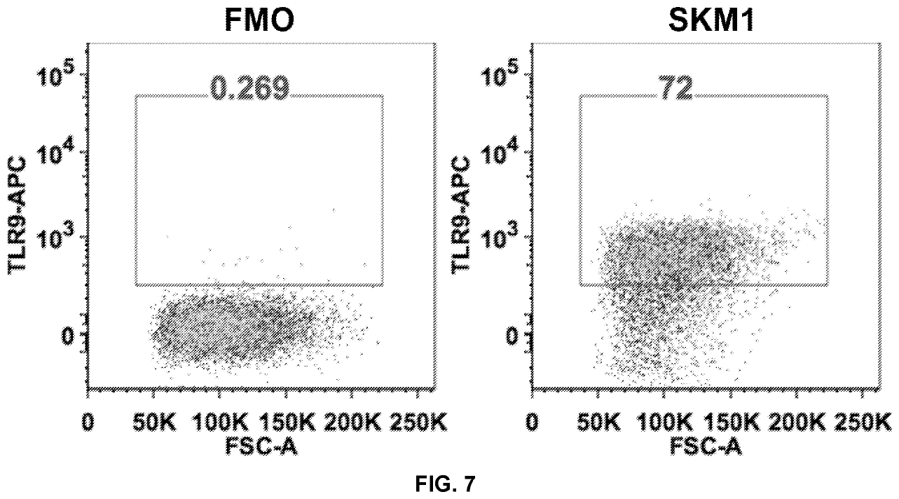

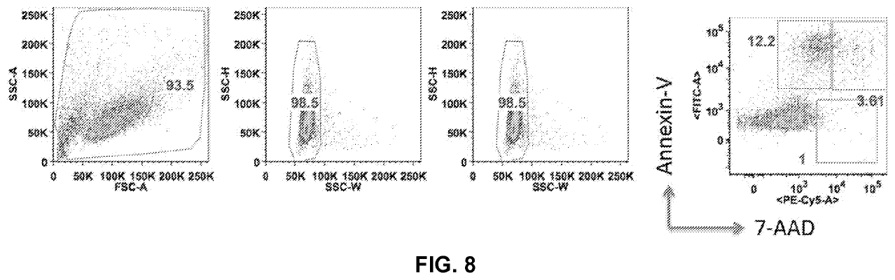

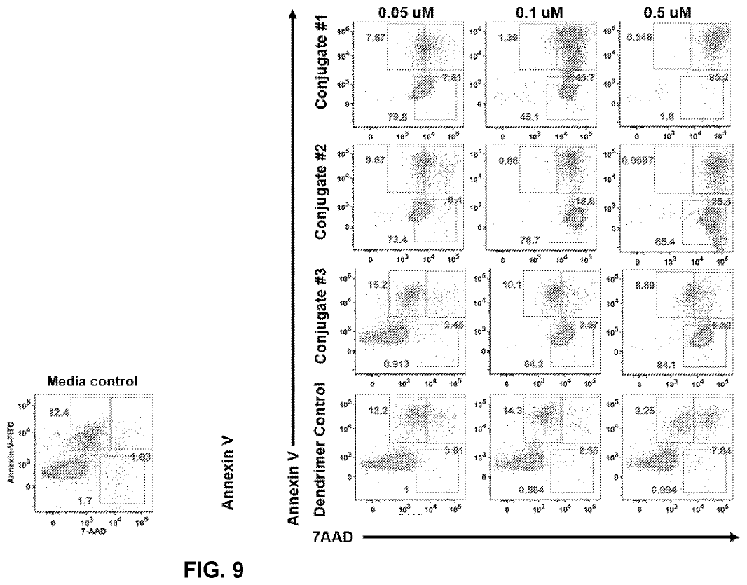

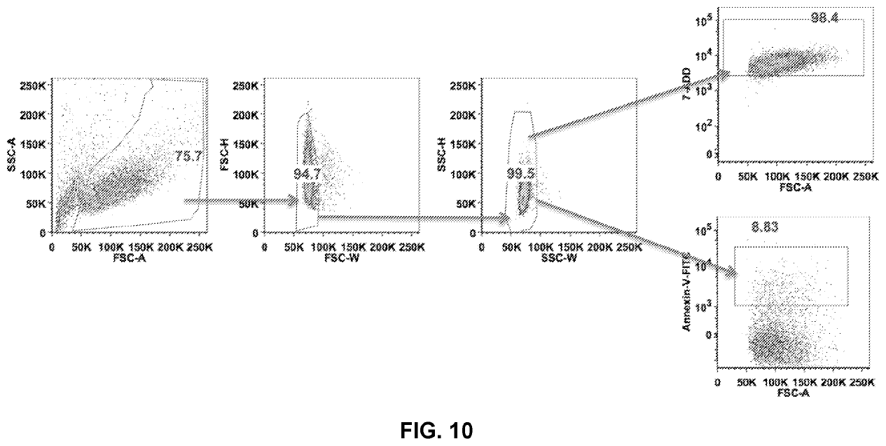

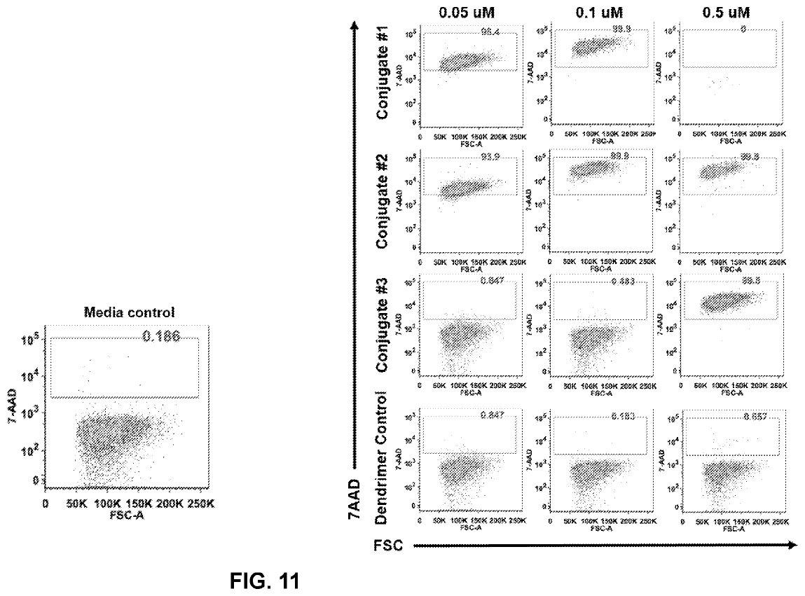

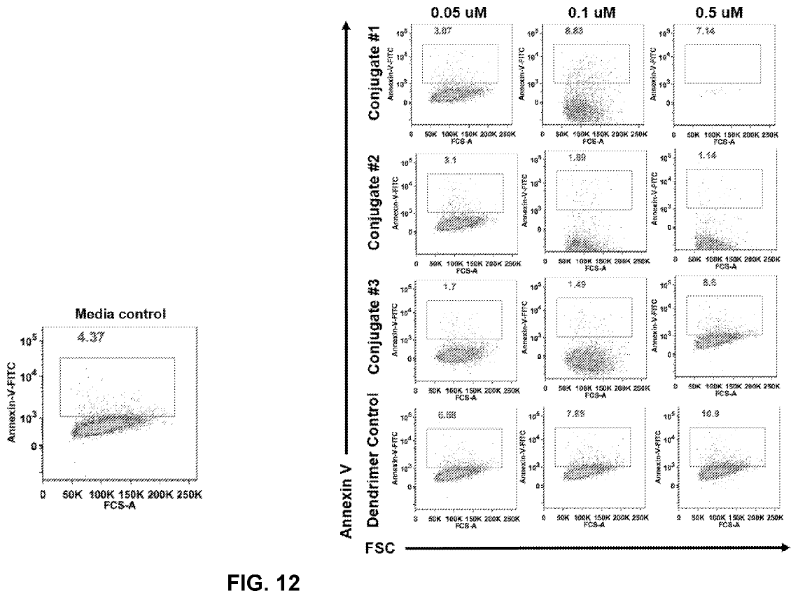

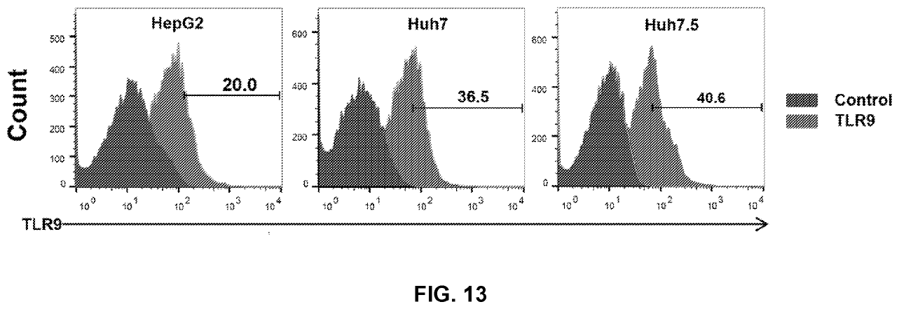

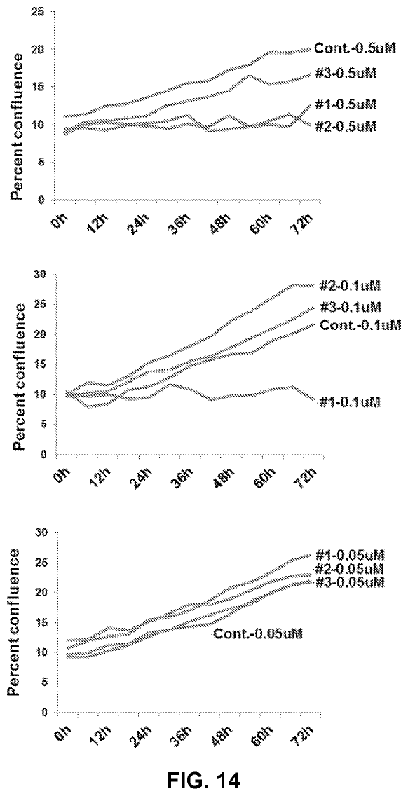

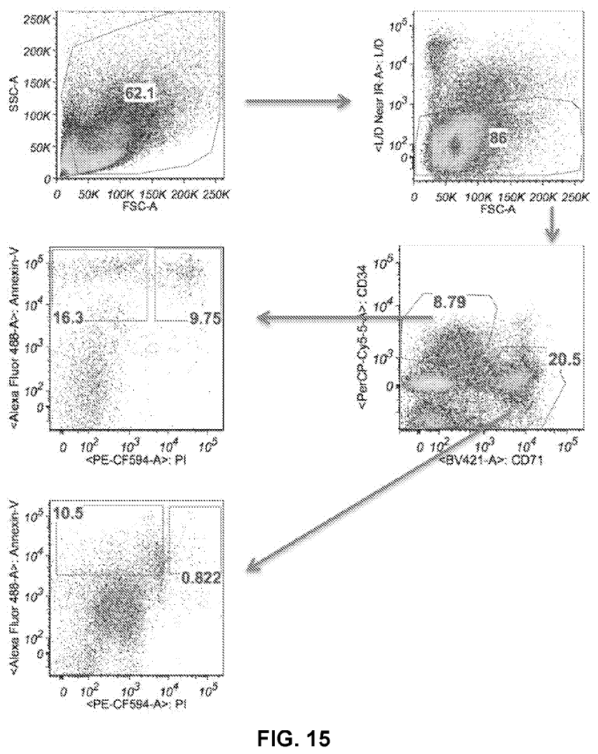

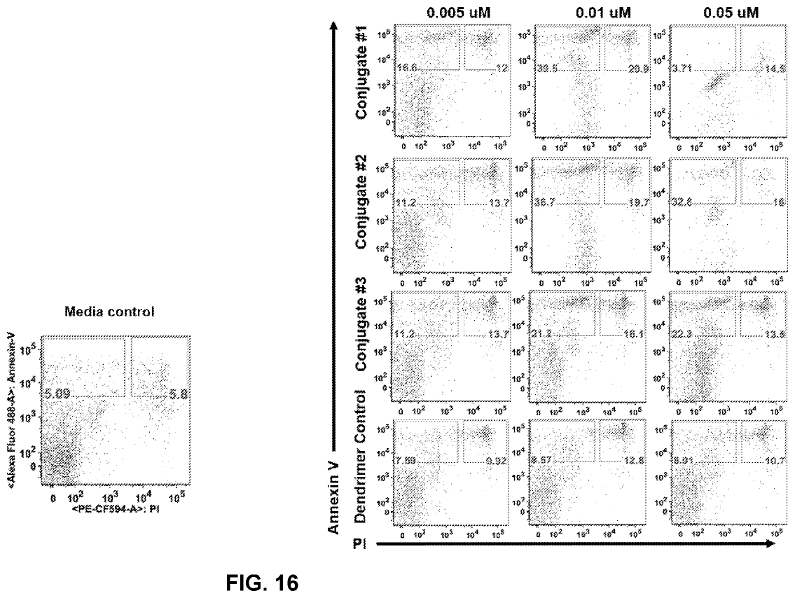

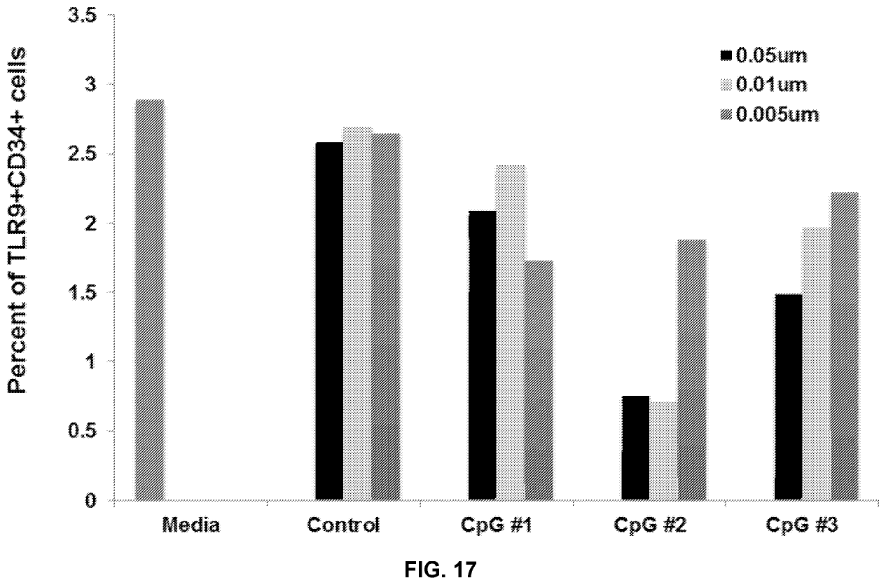

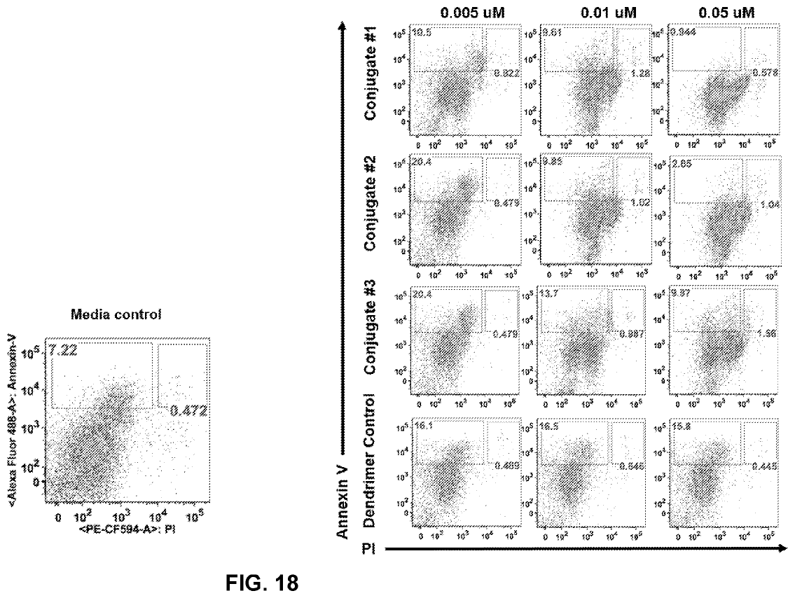

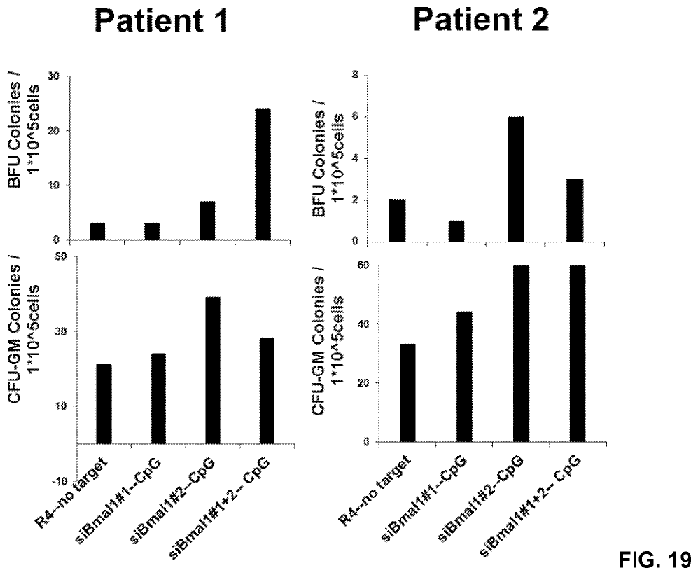

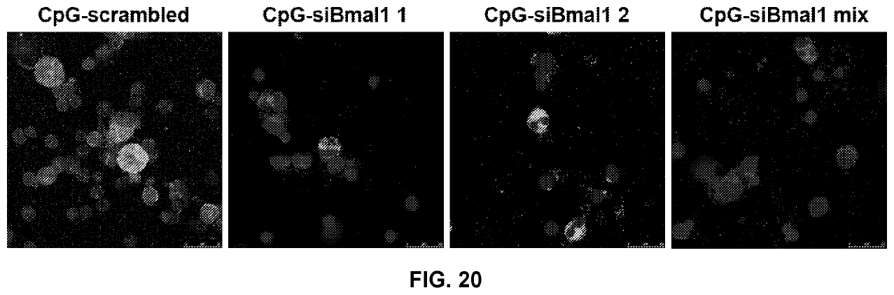

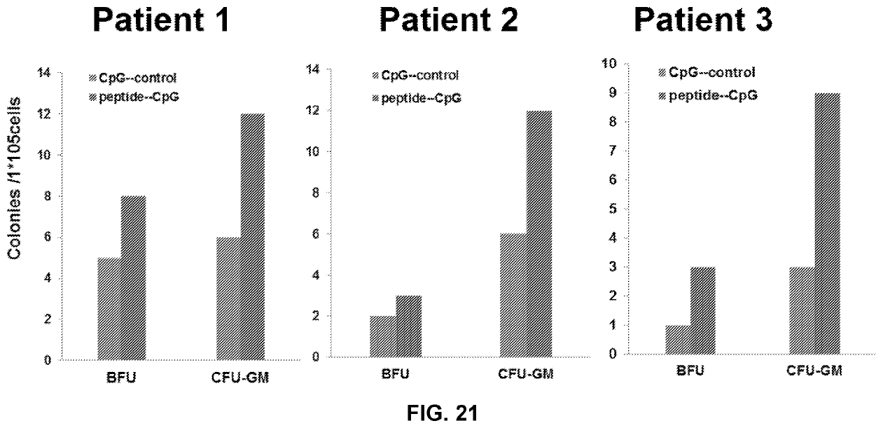

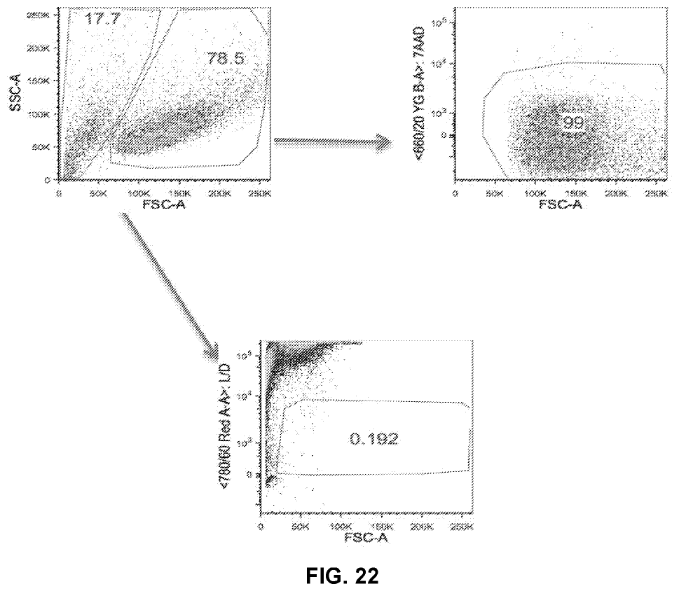

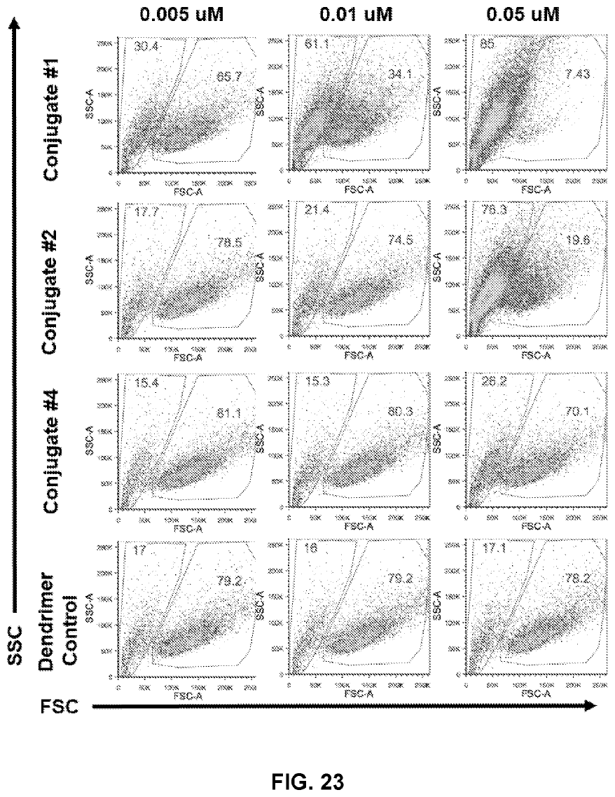

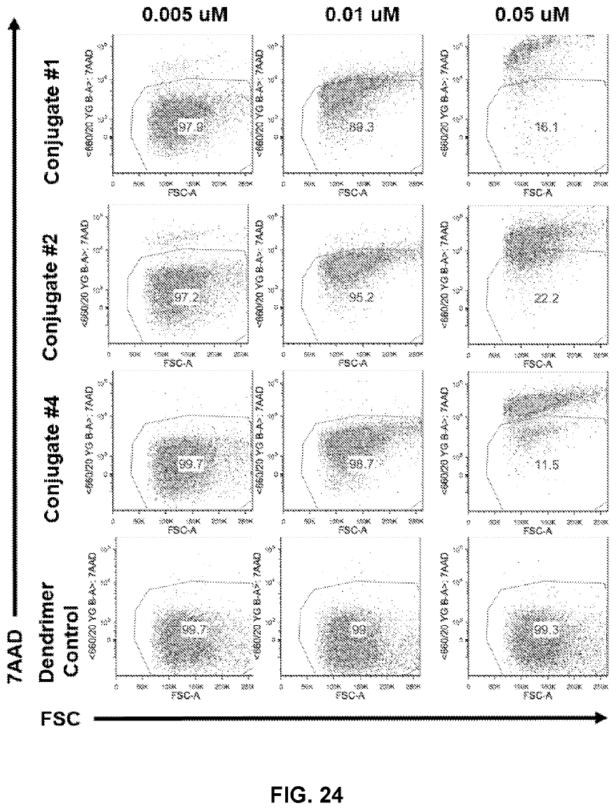

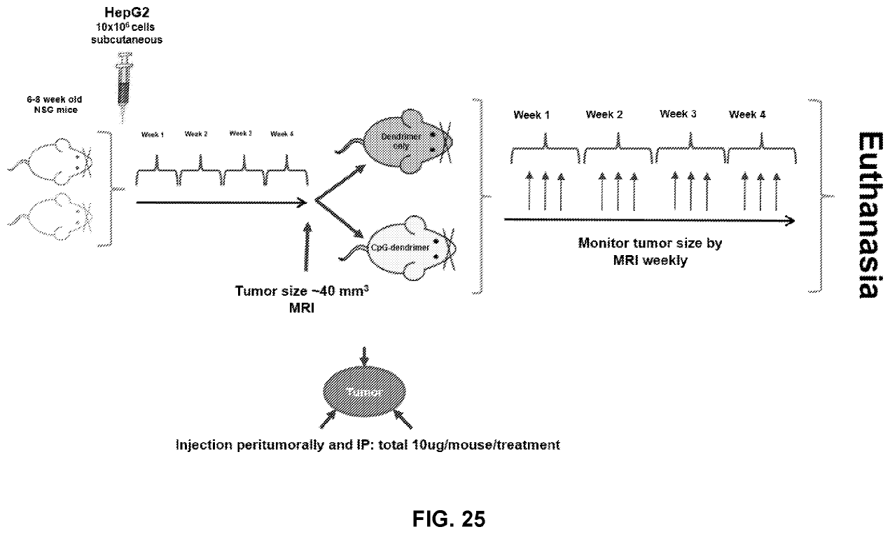

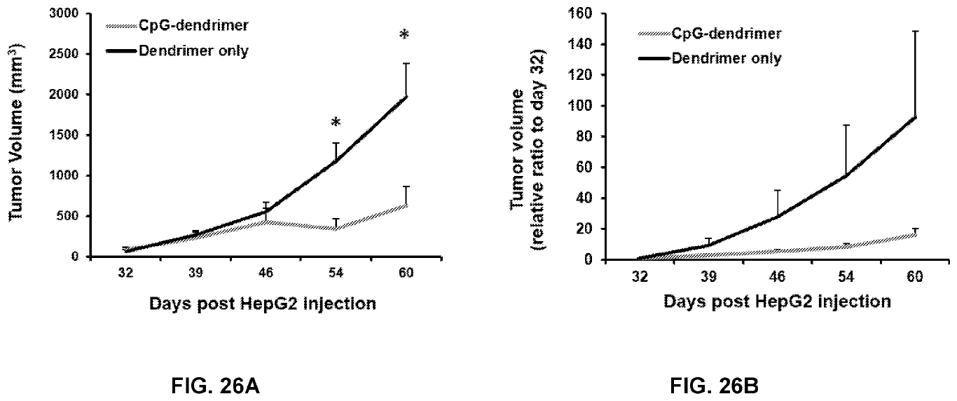

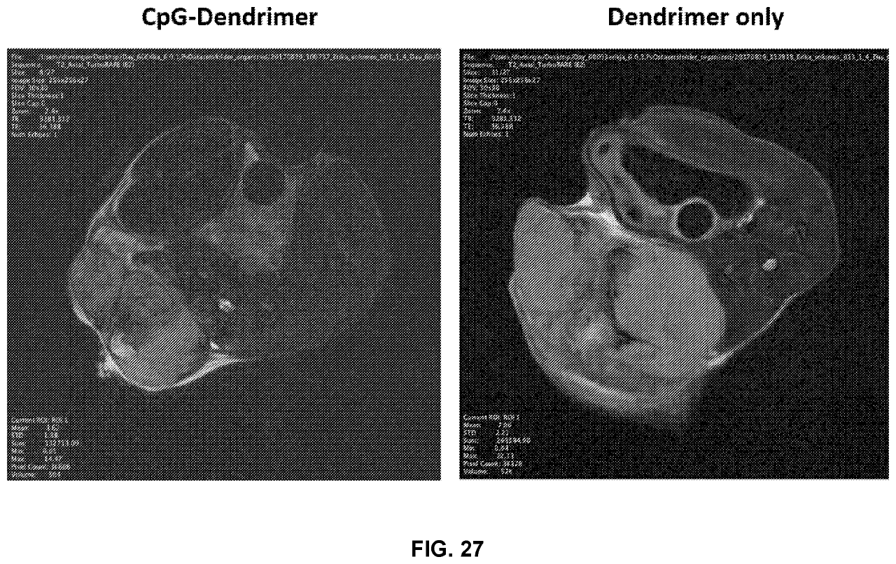

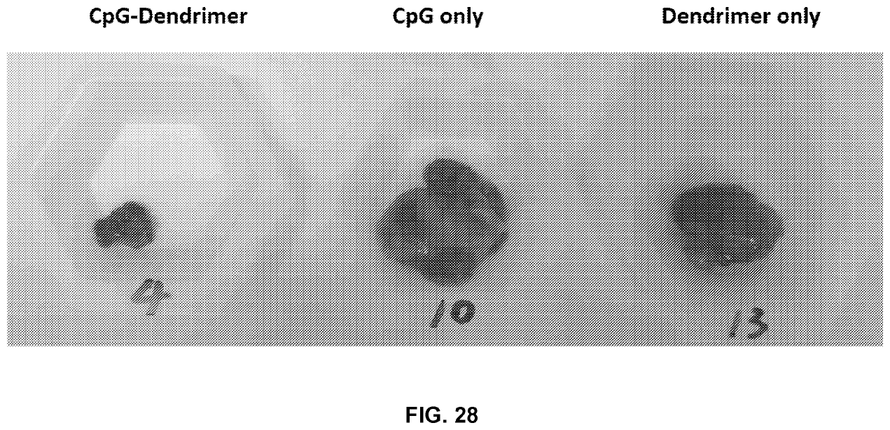

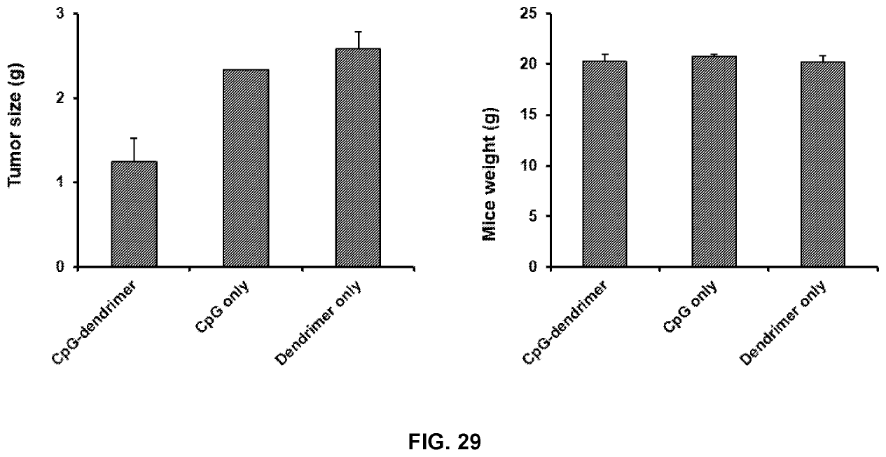

The biodegradable polymeric backbone can comprise polymerized amino acids, such as polymerized L-lysine (PLK or poly-L-lysine), polymerized D-lysine, polymerized L-glutamic acid (PLE), polymerized D-glutamic acid, polymerized aspartic acid (PASA), and polycarbohydrate such as polysaccharides, or a combination thereof. Some of the polymer examples are illustrated in FIG. 6B. Polymer segments comprising linear poly-L-lysine, branched poly-L-lysine, linear poly-L-glutamic acid, branched poly-L-glutamic acid, linear polyaspartic acid, branched polyaspartic acid, or a combination thereof, can be preferred. Polymer segments comprising polysaccharide can also be suitable. A copolymer polymerized from a combination of lysine, glutamic acid, and/or aspartic acid can also be suitable. In one example, the biodegradable polymeric backbone comprises polymerized modified or unmodified amino acids, modified or unmodified polycarbohydrate, or a combination thereof. In another example, the modified or unmodified amino acids comprise polymerized modified or unmodified L-lysine (PLK or poly-L-lysine), polymerized modified or unmodified D-lysine, polymerized modified or unmodified L-glutamic acid (PLE), polymerized modified or unmodified D-glutamic acid, polymerized modified or unmodified aspartic acid, or a combination thereof. As used herein, the term PLK or polylysine includes polymerized modified or unmodified L-lysine or D-lysine, the term PLE or polyglutamic acid includes polymerized modified or unmodified L-glutamic acid or D-glutamic acid, polymerized modified or unmodified aspartic acid, unless specifically defined.

The biodegradable polymeric backbone can comprise a polymerized sugar, disaccharide, polysaccharide, or a combination thereof, and the biodegradable bond is a glycosidic bond. The biodegradable polymeric backbone can comprise disaccharide that contains glucose, fructose, or a combination thereof, starch, cellulose, modified polysucrose, dextran, modified dextran, hyaluronic acid, or a combination thereof.

The cationic polymer components, which can be covalently attached to biodegradable polymeric backbone, can comprise polymerized ethyleneimine (PEI), polypropyleneimine (PPI), polyamidoamine (PAMAM), polylysine, polyarginine, and/or a combination thereof. Each of the cationic polymer components can be independently a linear polymer, a branched polymer, a hyperbranched polymer, a graft polymer, a block polymer, a dendrimer, or a combination thereof. The term “polymer” or “copolymer” used herein throughout this application refers to polymer polymerized from same monomer (homopolymer), two or more different monomers (copolymer), or a combination thereof, unless specifically specified. Also as mentioned herein, each of the cationic polymer components can be the same or different and a dendrimer can be symmetric or asymmetric.

The biodegradable polymeric backbone can comprise in a range of from 2 to 1,000 polymerized lysine, polymerized glutamic acid, or a combination thereof. It is preferred that the biodegradable polymeric backbone comprises polymerized lysine, polymerized glutamic acid, polymerized aspartic acid, or a combination thereof. The biodegradable polymeric backbone can be the backbone of the biodegradable polymer. The biodegradable polymeric backbone can comprise in a range of from 2 to 1,000 lysine residues in one example, 2 to 800 lysine residues in another example, 2 to 600 lysine residues in yet another example, 2 to 400 lysine residues in yet another example, 2 to 200 lysine residues in another example, and 2 to 100 lysine residues in another example. The biodegradable polymeric backbone can comprise in a range of from 2 to 1,000 glutamic acid residues in one example, 2 to 800 glutamic acid residues in another example, 2 to 600 glutamic acid residues in yet another example, 2 to 400 glutamic acid residues in yet another example, 2 to 200 glutamic acid residues in another example, and 2 to 100 glutamic acid residues in another example. L-amino acids can be preferred.

Each of the cationic polymer components can independently have a molecular weight (MW) in a range of from about 100 to about 100,000. The cationic polymer component can have a molecular weight (MW) in a range of from about 100 to about 100,000 in one example, about 100 to about 80,000 in another example, about 100 to about 60,000 in yet another example, about 100 to about 40,000 in yet another example, about 100 to about 35,000 in yet another example, about 100 to about 30,000 in yet another example, about 100 to about 25,000 in yet another example, about 100 to about 15,000 in yet another example, about 100 to about 10,000 in yet another example, and about 100 to about 5,000 in yet another example. The cationic polymer component having lower molecular weight, such as from about 100 to about 30,000, about 100 to about 25,000, about 100 to about 10,000 or about 100 to about 5,000 can be preferred.

The cytotoxic nanoparticles comprising the CpG-linked cationic polymer can have particle size measured in mean diameter in a range of from 1 nm to 1000 nm. The nanoparticles can be dispersible in aqueous solutions. The nanoparticles can have a size in a range of from 1 nm to 1,000 nm in an example, 5 nm to 1000 nm in another example, 10 nm to 1000 nm in yet another example, 20 nm to 1000 nm in yet another example, 30 nm to 1000 nm in yet another example, and 50 nm to 1000 nm in yet another example. The nanoparticles can also have a size in a range of from 1 nm to 900 nm in an example, 1 nm to 700 nm in another example, 1 nm to 500 nm in yet another example, 1 nm to 300 nm in yet another example, 1 nm to 200 nm in yet another example, 1 nm to 100 nm in yet another example, 1 nm to 50 nm in yet another example, 1 nm to 20 nm in yet another example, and 1 nm to 10 nm in yet another example. The nanoparticles can have a mean diameter of 1.5 to 50 nm.

The cytotoxic nanoparticles can be formed in solutions. The nanoparticles can be lyophilized for stability and long-term storage. The lyophilized nanoparticles comprising the CpG-linked cationic polymer can be reconstituted with an aqueous solution or an organic solvent. In one example, the lyophilized nanoparticles are reconstituted in an aqueous solution, such as saline or other pharmaceutically acceptable solutions.

In one example, the CpG-NH₂ can be reacted with Traut's reagent follow with purification to produce CpG-SH. The Polymer, for example, a branched PEI polymer or a PAMAM dendrimer, can react with a hetero functional linker such as Maleimide (MAL)-PEG-NHS ester and purified to produce polymer-MAL. The polymer-MAL then reacts with CpG-SH to generate the final conjugate. In another example, polymer reacts with Traut's reagent, then is purified, following with a reaction with CpG-SMCC to generate the final conjugate.

Each of the CpG molecules can be conjugated with one or more polymers. In one example, one CpG is linked to two polymers via a multi-valent linker. In one example, the bivalent linker disclosed above can be modified with multi-amino, multi-imino, carboxyl, or —SH, or —OH functional groups to produce a multi-valent linker, which allows the attachment of multiple polymers per CpG (FIG. 6B).

The TLR9 targeting ligand can also comprise other nucleic acids, for example, an unmethylated GpC oligodeoxynucleotide, a random oligodeoxynucleotide or a combination thereof. In examples, the molecule can comprise GpC-linked cationic polymer, a random oligodeoxynucleotide-linked cationic polymer, or a combination thereof. The term “GpC”, “GpC oligodeoxynucleotide” or “GpC ODN” used herein refers to an oligodeoxynucleotide comprising at least one GpC motif, which contains unmethylated guanine-cytosine dinucleotide linked by a phosphate bond. In one example, a GpC can have a sequence T*C*C*A*T*G*A*G*C*T*T*C*C*T*G*A*T*G*C*T* (SEQ ID NO:2). The term “random oligodeoxynucleotide” refers to a nucleic acid that is a polymeric form of deoxynucleotides with a random sequence. The GpC oligodeoxynucleotide or the random oligodeoxynucleotide can be conjugated to the dendrimers disclosed herein. The molecules comprising GpC-linked cationic polymer, a random oligodeoxynucleotide-linked cationic polymer, or a combination thereof can be produced with the conjugation process disclosed herein for the production of the GpC-linked cationic polymer. Cytotoxicity was also observed on the molecules that comprise a GpC-linked dendrimer, such as a GpC-PAMAM, or a random oligodeoxynucleotide-linked dendrimer PAMAM. Not wishing to be bound by a particular theory or mechanism, applicants postulate that dendrimer linked GpC or random oligodeoxynucleotide may also have potential TLR9-targeting effect.

### Pharmaceutical Composition

Also disclosed is a pharmaceutical composition comprising a molecule disclosed herein in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Methods of Treatment

Also disclosed is a method for treating a TLR9-expressing cancer, such as a myelodysplastic syndrome (MDS), in a subject by administering to the subject a therapeutically effective amount of the disclosed pharmaceutical composition. The method can further involve administering to the subject lenalidomide, or an analogue or derivative thereof. FIG. 4 identifies other cancers, such as lung and breast cancers, that have increased TLR9 expression. FIG. 5 shows TLR9 protein expression in a variety of tumor tissues. For example, cancers of the skin, esophagus, colon, rectum, liver, lung, and uterus have been shown to have increased TLR9 protein expression.

A representative but non-limiting list of cancers include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some cases, the method further involves assaying a biopsy sample from the subject for TLR9 expression prior to treatment. This can be done using routine methods, such as immunodetection methods. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for a TLR9-expressing cancer. Thus, the method can further comprise identifying a subject at risk for a TLR9-expressing cancer prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

With the purpose of using the high surface expression of the receptor TLR9 in the surface of stem cells, CpG, a cognate danger-associated molecular pattern (DAMP) recognized by TLR9, was used as a target for internalization of an amphipatic lytic peptide capable of lysis only when internalized. This allows the targeted killing of malignant TLR9+ hematopoietic stem cells in cancer.

A CpG sequence T*C*C*A*T*G*A*C*G*T*T*C*C*T*G*A*T*G*C*T* (SEQ ID NO:1) modified with thioate linkages (*=phosphorothioate linkage) was used. The initial attempt was by linking the CpG and the lytic peptide through the use of a Click-It reaction as demonstrated in FIG. 1. However, this reaction might be less stable, even though the initial reaction performed with CpG-lytic prepared this way has already shown effectivity in reducing TLR9+ stem cells. The second strategy involved the use of a C6 amino-SMCC-Cys linker as shown in FIG. 2. The CpG sequence is fully phosphothioated in order to increase stability of the CpG moiety.

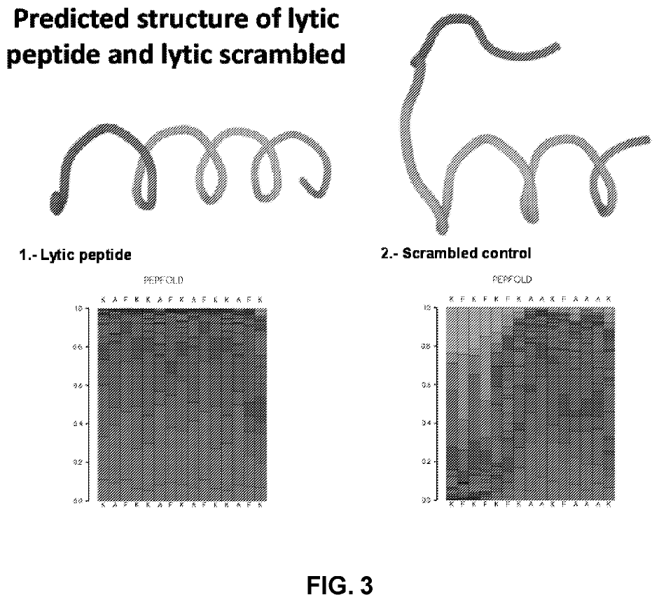

FIG. 2 also shows the sequence of a new lytic peptide and a scrambled sequence that was used as control. An important thing to note is that the sequence itself is important, because of the amphitatic nature of the residues, but its main function relies on the helical conformation of the peptide. Hence, as a control the same sequence of peptides was used but was looped through a 3D structure predictor until a scrambled sequence was found were the helical structure was disrupted (FIG. 3).

### Example 2: Polymer and Oligodeoxynucleotide Conjugate

Materials were purchased from Dendritech, Sigma-Aldrich, Quanta BioDesign, Bio-Synthesis Inc., Integrated DNA Technologies Inc., PolySciences Inc., ThermoFisher or other appropriate commercial sources.

Preparation of functionalized CpG: 272.3 nmol of CpG having the sequence shown in Example 1 with amine functional group was dissolved in 2.7 mL of borate buffer and was reacted with 194.5 μL of Traut's reagent (28 mM in borate) at room temperature for 1 hour. The reaction mixture was then precipitated with 292 μL of 3M sodium acetate and 8.0 mL of ethanol. The functionalized CpG was separated by centrifuge at 14,000 g for 60 minutes as a pallet. The pallet was then air dried and stored at −20° C. Upon reconstitution, a phosphate buffer was added to product a functionalized CpG at a concentration of 1.8 mg/m L.

About 1.2 mg of PAMAM polymer (E3) solution in methanol (17.5%) was added with 113 μL of a maleimide poly(ethylene glycol)N-hydroxysuccinimide ester linker (MAL-PEG linker), in PBS (2%) and incubated at 30° C. for 15 minutes. The reaction mixture was then purified with Gel Filtration Chromatography (GFC) and collected in 2.09 mL in a phosphate buffer. Then, 0.32 mL of the activated PAMAM E3 polymer was mixed with 23.5 nmol of the functionalized CpG solution prepared above with 18 μL of 1M sodium carbonate. The reaction mixture was incubated at room temperature for 1 hour, and then purified with ultra-filtration at 14,000 g for 30 min. The solution was freeze-dried to produce an E3 PAMAM-CpG conjugate (FIG. 6A).

About 67 mg of PAMAM polymer (E5) solution in methanol (27%) was added with 880 μL of a maleimide poly(ethylene glycol)N-hydroxysuccinimide ester linker (MAL-PEG linker), in PBS (1%) and incubated at 30° C. for 15 minutes. The reaction mixture was then purified with Gel Filtration Chromatography (GFC) and collected in 9.65 mL in a phosphate buffer. Then, 6.21 mL of the activated PAMAM E5 polymer was mixed with 182 nmol of the functionalized CpG solution prepared above in 289 μL of 1M sodium carbonate. The reaction mixture was incubated at room temperature for 1 hour, and then purified with ultra-filtration at 14,000 g for 30 min. The solution was freeze-dried to produce PAMAM-linker-CpG conjugate PAMAM-MAL-PEG-CpG (FIG. 6A).

With a similar process, PAMAM CpG with a SMCC linker was prepared by reacting the aforementioned CpG modified with a C6 amino SMCC linker at its 3′ end and the PAMAM E5 polymer described above further modified with thiol functional groups. The product was an E5 PAMAM-CpG conjugate (FIG. 6A).

With similar process, a GpC oligodeoxynucleotide or a random oligodeoxynucleotide can be conjugated to any one of the dendrimers disclosed herein. For example, the dendrimer can comprise PAMAM, PEI, or a combination thereof. A GpC oligodeoxynucleotide (SEQ ID NO:2) and a random oligodeoxynucleotide was individually conjugated to the PAMAM dendrimer as described above.

### Example 3: Biodegradable Polymer PEI-PLK

Cationic biodegradable polymer polyethyleneimine-polylysine (PEI-PLK) was prepared by reacting 145 mg of branched polyethyleneimine (MW 1800 Dalton) in 4.03 g of DMSO with 1.304 g of Boc-Lys(Boc)-OSu in 4.32 g of DMSO at room temperature for 4 hours. The mixture was then precipitated, deprotected and then dissolved in DMSO to produce a lysine modified PEI (PEI-Lysine) that comprises multiple lysine residues on the polymer. One half of the PEI-lysine from the step above was mixed with 2.05 g of Boc-Lys(Boc)-OSu and diisopropylethylamine and reacted for 4 hours. The reaction mixture was precipitated and deprotected by reacting with methylenechloride and trifluoracetic acid at room temperature for 1 hour. The reaction mixture above was air dried with blowing nitrogen at 50° C. to produce the cationic biodegradable dendritic polymer polyethyleneimine modified with two layers of lysine (PLK2) having biodegradable lysine-lysine peptide bonds, herein referred to as Den(PEI-PLK2).

Dendrimers with 3 or more layers of lysine, Den(PEI-PLK3), Den(PEI-PLK4), etc., were prepared by repeating the Boc-Lys(Boc)-OSu reaction and deprotection steps described above.

CpG conjugates were prepared with the biodegradable polymers prepared above using the procedure described in Example 2 with selected linkers.

### Example 4: Biodegradable Polymer PEI-PLK-EI/PEI

One half of the Den(PEI-PLK2) prepared above was mixed with 1.79 g of bromoethylamine in methanol and 1.65 g of diisopropylethylamine, reacted at room temperature for 2 hours. The reaction mixture was precipitated with diethylether. The supernatant was removed. The precipitated contents were dialyzed against water. The resulted polymer water solution was rotary evaporated to dryness to produce a cationic dendritic polymer that has a branched PEI core modified with two layers of lysine (PLK2) and a plurality of lysine amine end groups modified with one or more ethylamine (EA), herein referred to as Den(PEI-PLK2-EI/PEI).

Same procedure was used to modify dendrimers with 3 or more layers of lysine, Den(PEI-PLK3), Den(PEI-PLK4), etc., to produce cationic biodegradable dendritic polymers that each has a branched PEI core modified with three or more layers of lysine and at least one lysine amine end group modified with one or more ethylamine (EA), herein referred to as Den(PEI-PLK3-EI/PEI) and Den(PEI-PLK4-EI/PEI), respectively.

CpG conjugates were prepared with the biodegradable polymers prepared above using the procedure described in Example 2 with selected linkers.

### Example 5: In Vivo Treatment Using CpG-Dendrimer Conjugates

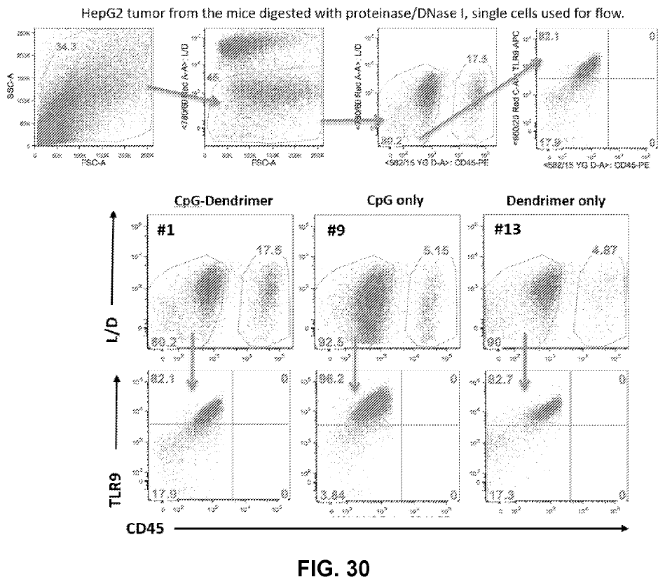

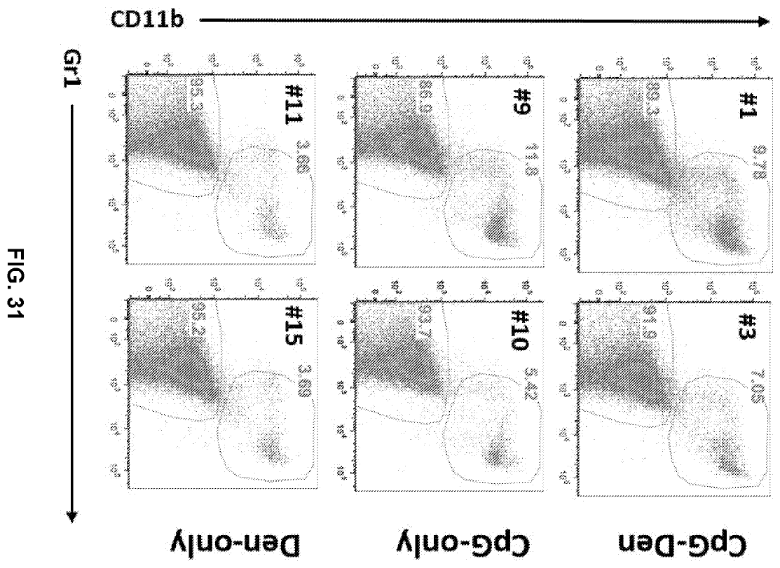

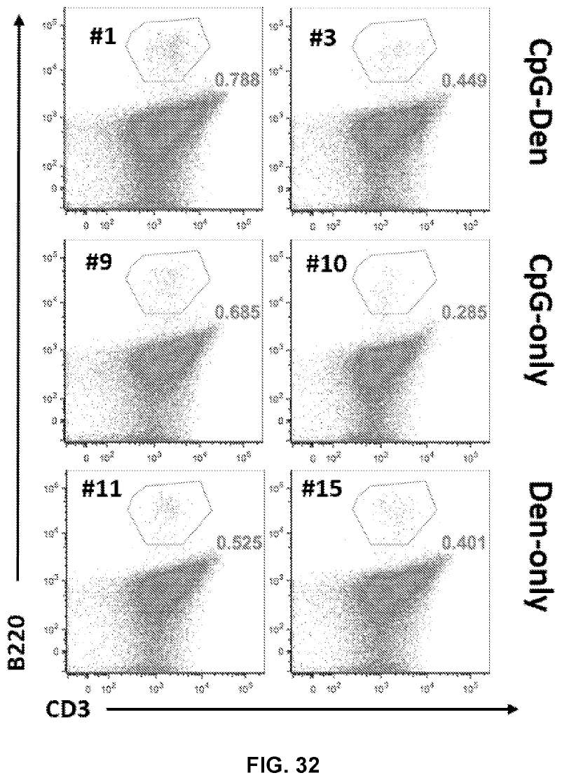

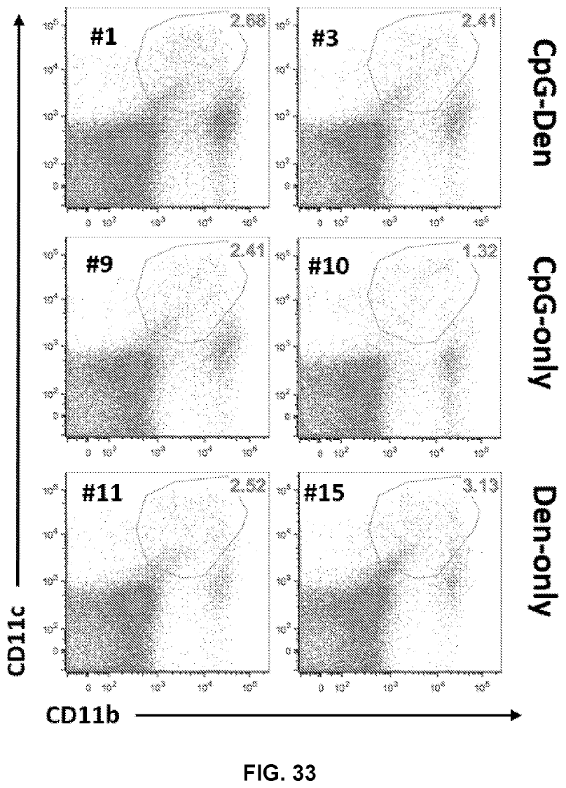

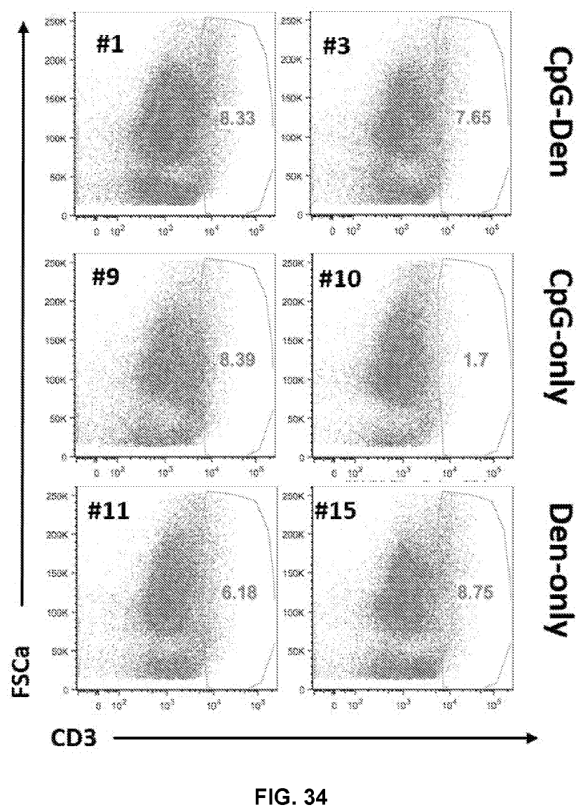

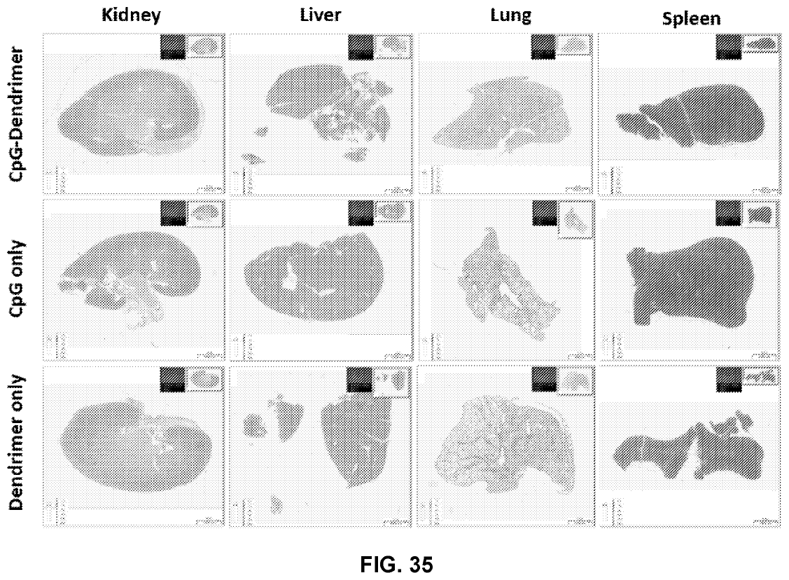

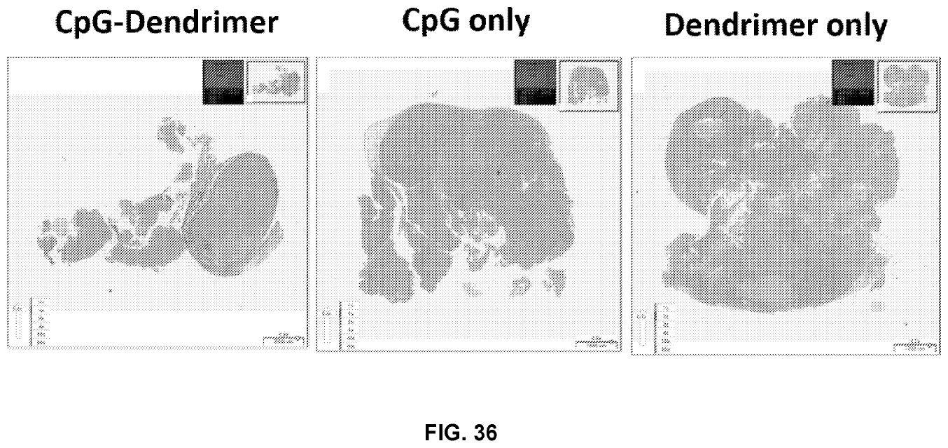

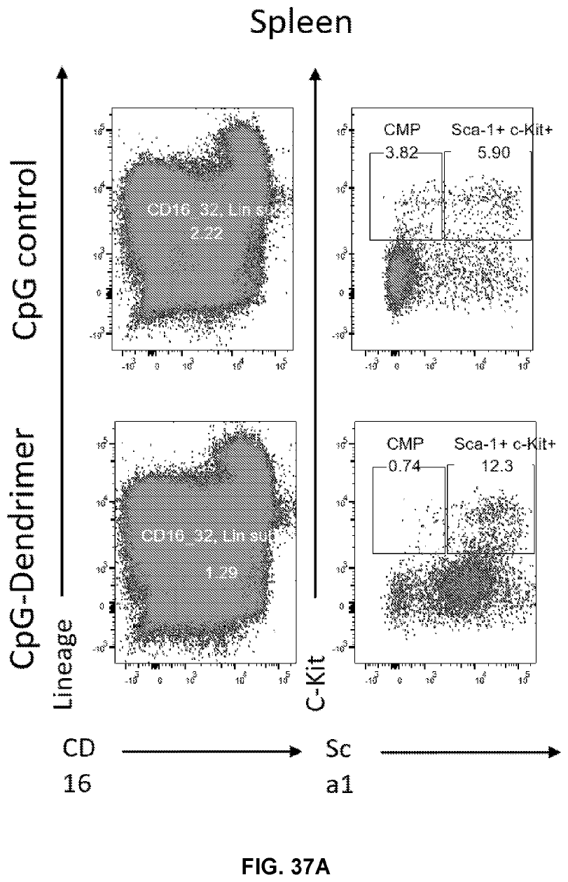

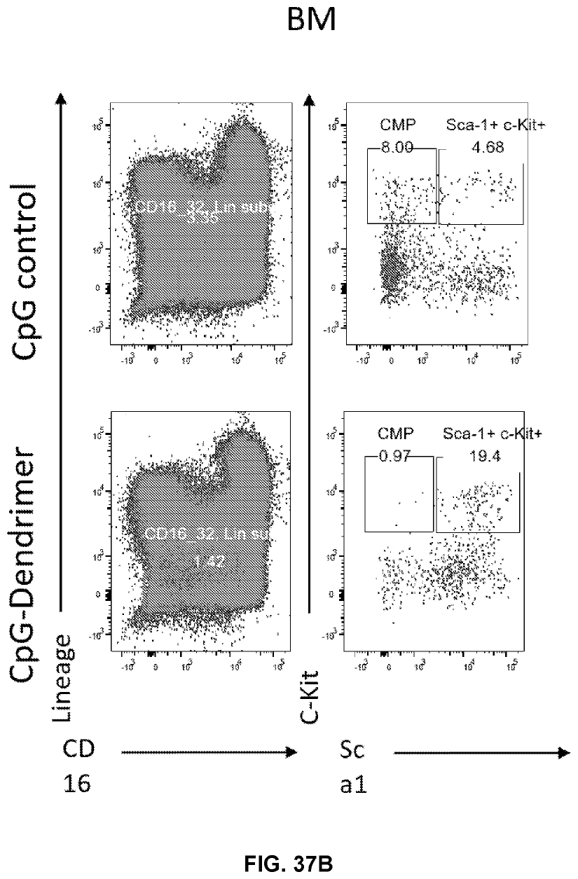

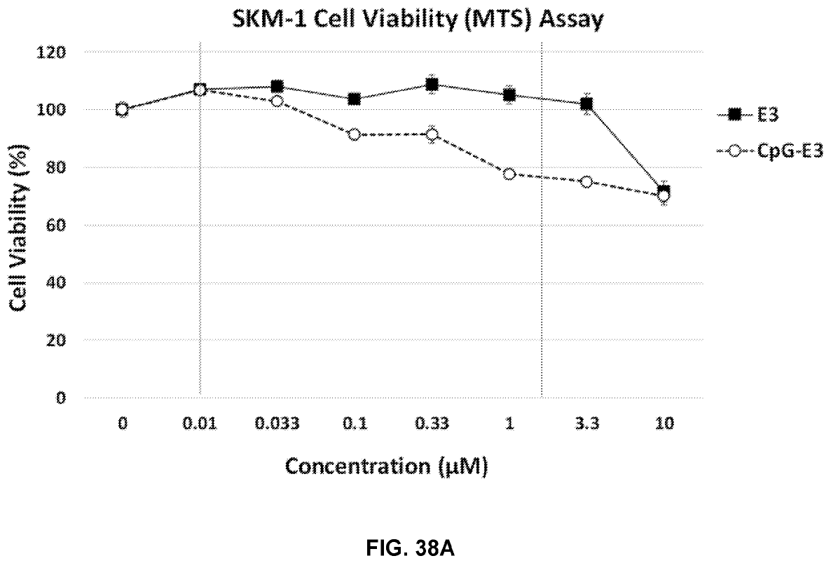

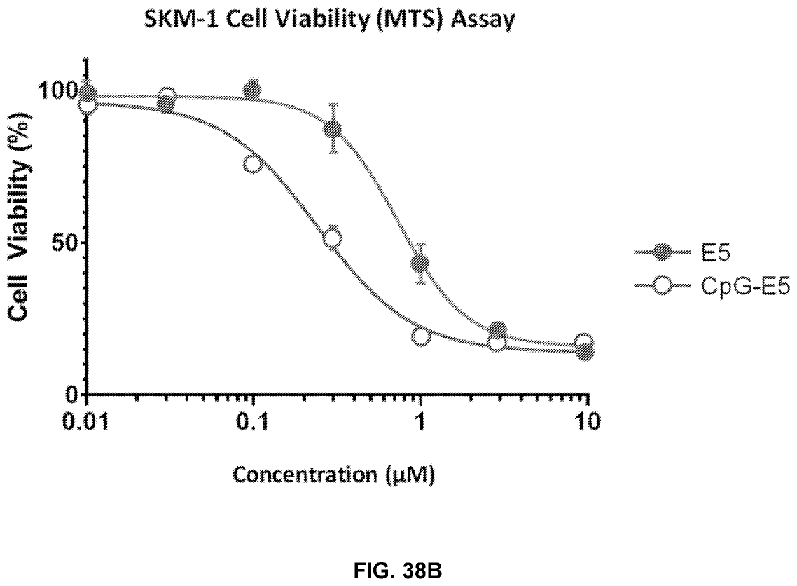

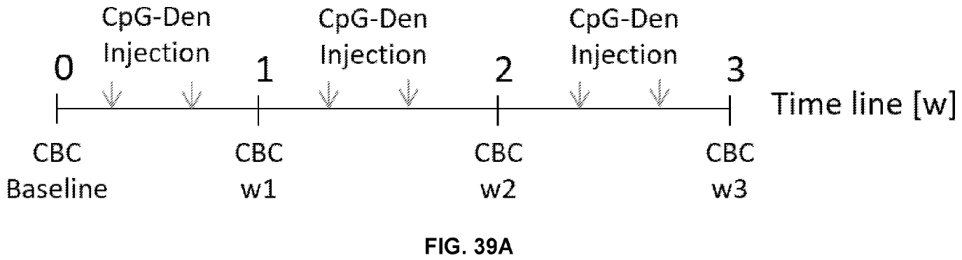

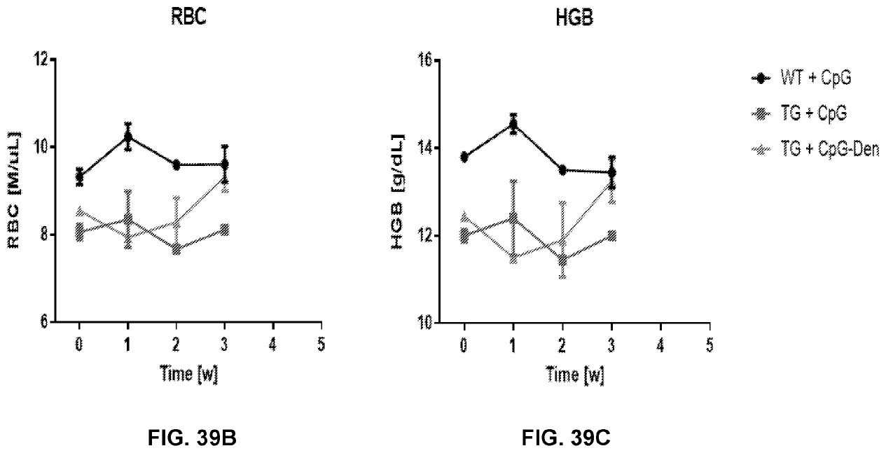

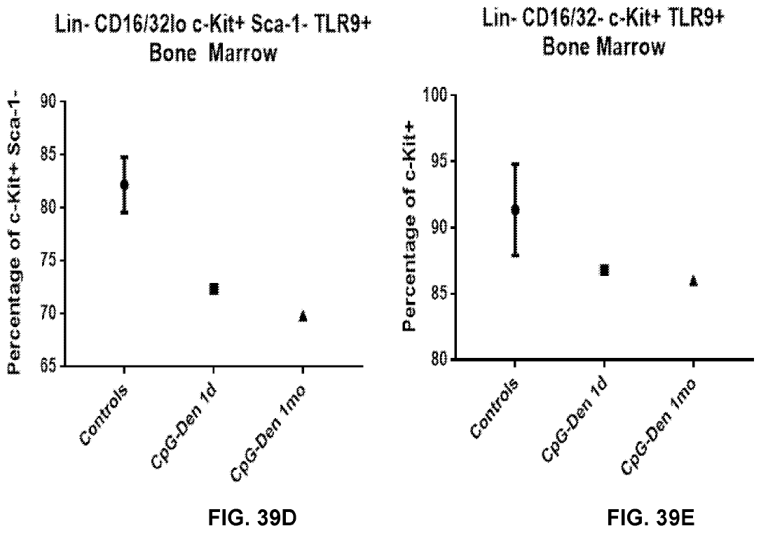

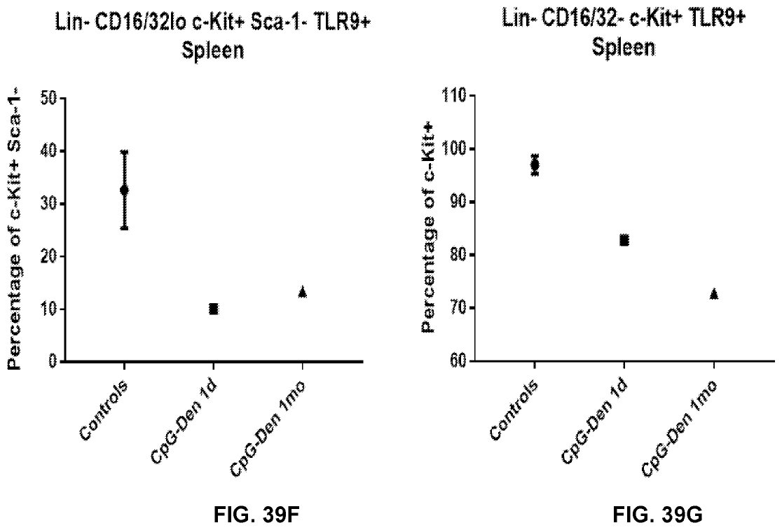

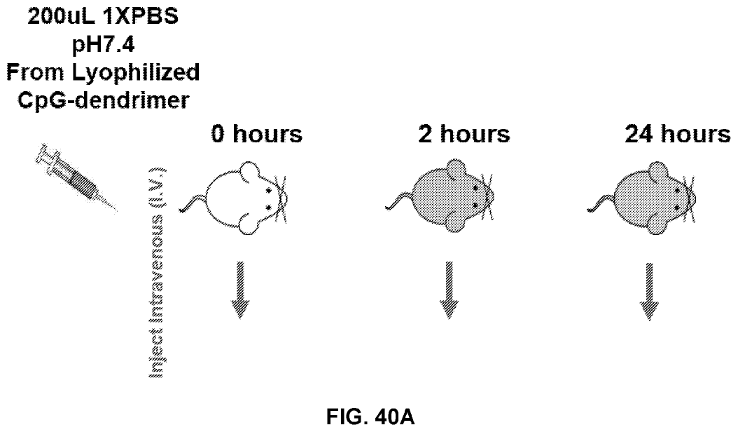

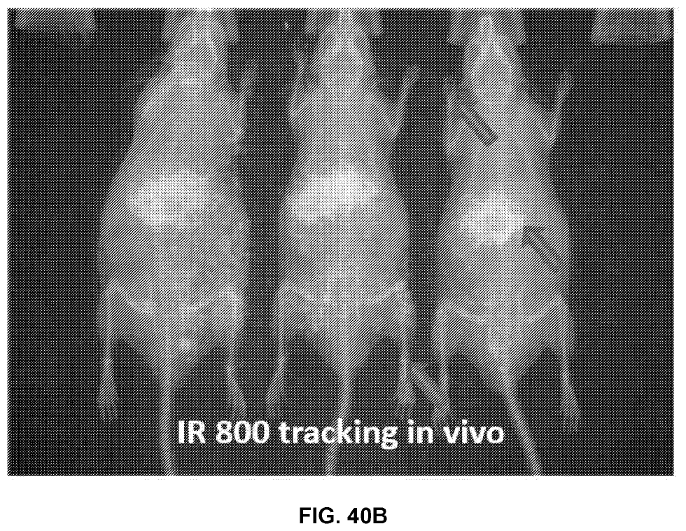

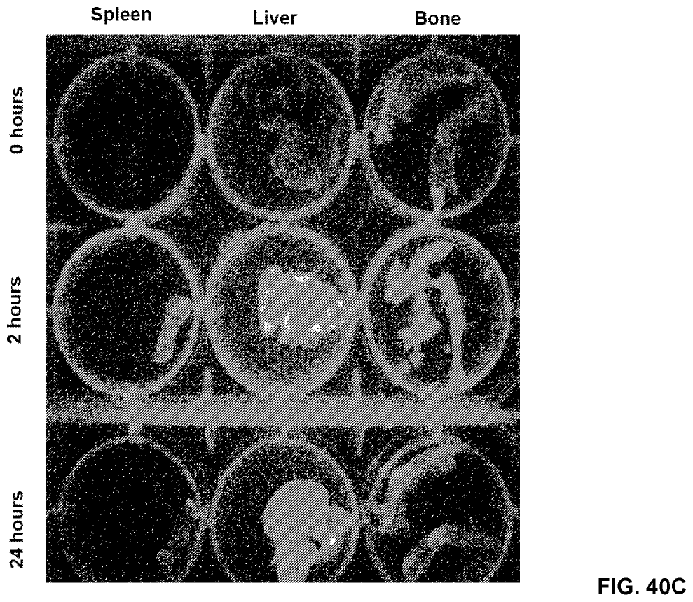

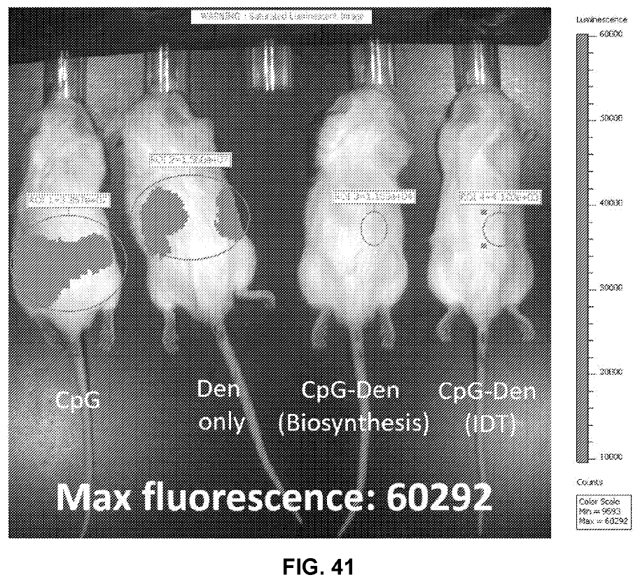

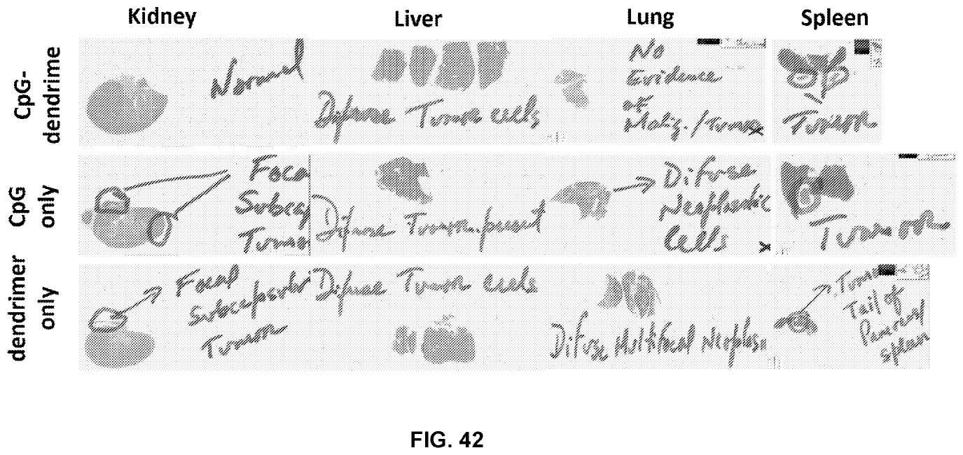

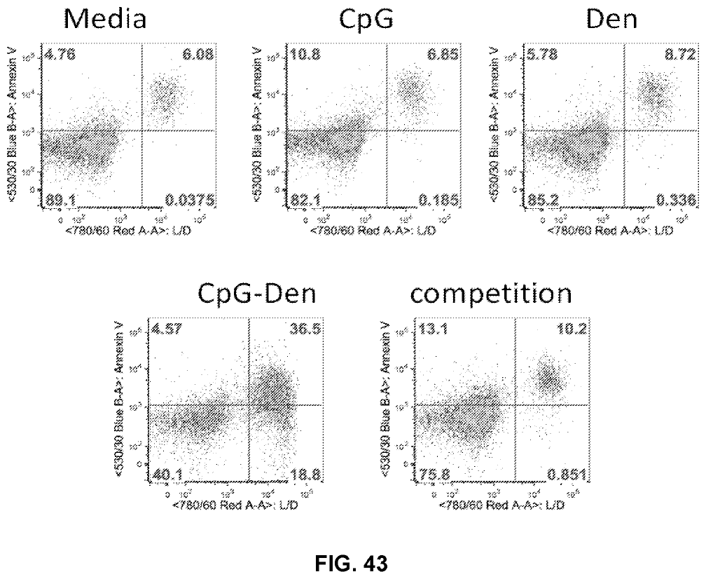

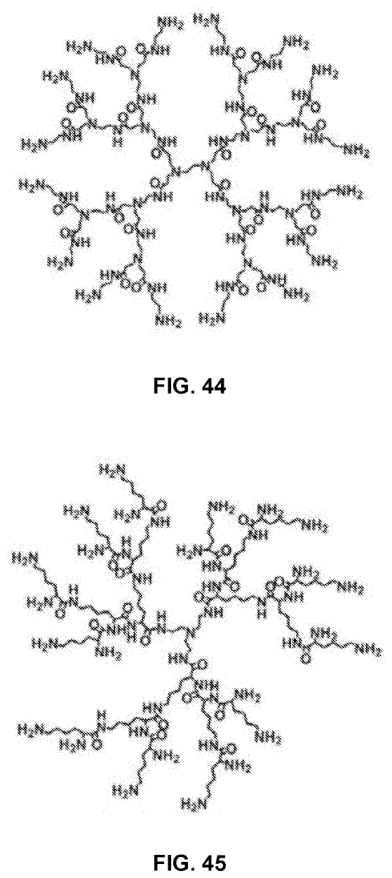

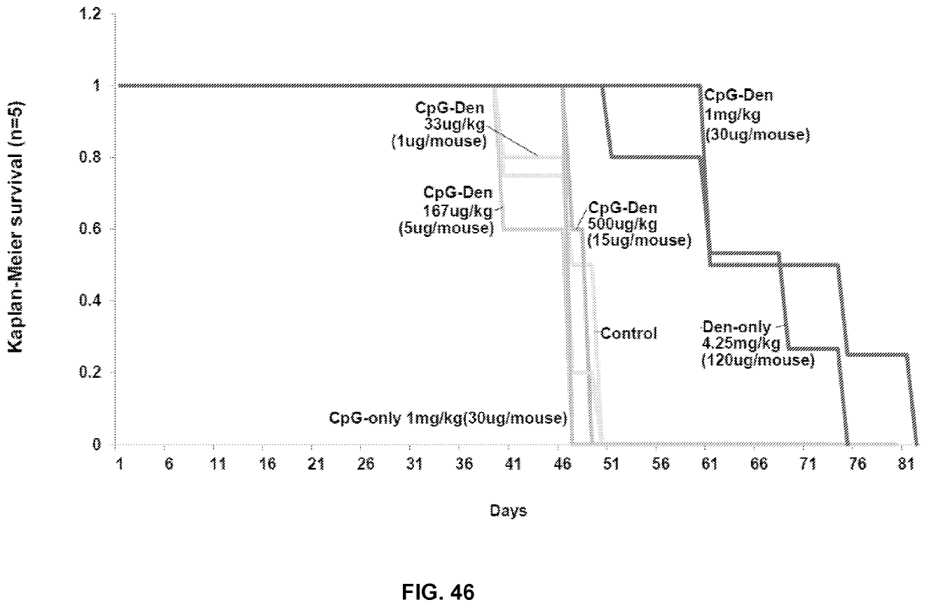

FIGS. 30 to 43 show in vivo effects of CpG-dendrimers.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A molecule comprising a toll like receptor-9 (TLR9) targeting ligand conjugated to a cytotoxic agent via a bivalent linker, wherein the cytotoxic agent comprises a cytotoxic nanoparticle.

2. The method of claim 1, wherein the cytotoxic nanoparticle comprises a cationic polymer, wherein said ligand is covalently conjugated to said cationic polymer via said bivalent linker.

3. The molecule of claim 1, wherein the TLR9 targeting ligand is an unmethylated CpG oligodeoxynucleotide, or an analogue or derivative thereof that binds TLR9.

4. The molecule of claim 1, wherein the TLR9 targeting ligand comprises an unmethylated GpC oligodeoxynucleotide, a random oligodeoxynucleotide or a combination thereof.

5. The molecule of claim 1, wherein the bivalent linker comprises a C6 amino-SMCC-Cys linker.

6. The molecule of claim 1, wherein the cytotoxic nanoparticle has a mean diameter of 1.5 to 50 nm.

7. The molecule of claim 1, wherein the cytotoxic nanoparticle comprises a symmetrical dendrimer, an asymmetrical dendrimer, a linear and/or branched homopolymer, a block copolymer, a graft copolymer, a random copolymer, or a combination thereof.

8. The molecule of claim 1, wherein the cytotoxic nanoparticle comprises a biodegradable polymer comprising a biodegradable polymer backbone comprising at least a biodegradable bond and two or more cationic polymer components, wherein each of the cationic polymer components is attached to the biodegradable polymeric backbone covalently separated by at least one of the biodegradable bond, wherein the CpG is covalently conjugated to the biodegradable polymer via the bivalent linker.

9. The molecule of claim 8, wherein the biodegradable polymeric backbone comprises polymerized modified or unmodified amino acids, modified or unmodified polycarbohydrate, or a combination thereof.

10. The molecule of claim 9, wherein the modified or unmodified amino acids comprise polymerized modified or unmodified L-lysine (PLK or poly-L-lysine), polymerized modified or unmodified D-lysine, polymerized modified or unmodified L-glutamic acid (PLE), polymerized modified or unmodified D-glutamic acid, polymerized modified or unmodified aspartic acid, or a combination thereof.

11. The molecule of claim 1, wherein the cytotoxic nanoparticle comprises a poly(amidoamine) (PAMAM) dendrimer.

12. The molecule of claim 1, wherein the cytotoxic nanoparticle is loaded with a cytotoxic agent.

13. A pharmaceutical composition comprising the molecule of claim 1 in a pharmaceutically acceptable carrier.

14. A method for treating a TLR9-positive cancer in a subject, comprising administering to the subject a therapeutically effective amount of the pharmaceutical composition of claim 13.

15. The method of claim 14, wherein the TLR9-positive cancer comprises a myelodysplastic syndrome (MDS).

16. The method of claim 15, wherein the TLR9-positive cancer comprises a hepatic cancer.

17. The method of claim 14, further comprising assaying a biopsy sample from the subject for TLR9 expression prior to treatment.
